(19) United States
(12) Patent Application Publication
(10) Pub. No.: US 2009/0130710 A1
(43) Pub. Date: May 21, 2009

(54) Enhancing vegetative protein production in transgenic plants using seed specific promoters

(76) Inventor: Allison Kermode, West Vancouver (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/914,112

(22) PCT Filed: May 9, 2006
(86) PCT No.: PCT/CA06/00747

§ 371 Date: May 19, 2008

Related U.S. Application Data
(60) Provisional application No. 60/679,433, filed on May 9, 2005.
(60) Provisional application No. 60/728,301, filed on Oct. 20, 2005.

(51) Int. Cl. C12P 21/04; C12N 5/04; C12N 15/00

(52) U.S. Cl. 435/69.1; 435/419; 435/320.1

(57) ABSTRACT

In various embodiments, the invention provides expression systems for heterologous protein expression in vegetative plant tissues, utilizing plant seed gene components that are adapted to orchestrate high levels of vegetative protein production. The expression systems may include host plant cells having recombinant genomes, and the plant cells may be maintained under protein expressing conditions, for example in tissue culture. The cells may be induced to express an ABD transcription factor, for example by transformation with a vector having a constitutive ABB expression cassette. The recombinant sequences in operative linkage may include an integrated expression promoter responsive to the ABI3 transcription factor, such as an arcelin gene promoter, a vicilin gene promoter and a napin gene promoter. A 5′ untranslated region may include a region of an ABA responsive plant seed gene or an AB 13 responsive plant seed gene. A plant secretion signal peptide coding sequence may be included. An integrated heterologous protein coding region, encoding a recombinant protein, may be provided in an open reading frame with the signal peptide coding sequence. A 3′ untranslated region may be provided having a polyadenylation signal.

### FIELD OF THE INVENTION

The invention is in the field of genetic engineering, specifically genetic manipulation of plant cells to facilitate heterologous protein production.

### BACKGROUND OF THE INVENTION

Transgenic plants or plant cells are potentially one of the most economical systems for large-scale production of recombinant proteins for industrial and pharmaceutical uses (Horn et al., 2004; Obermeyer et al., 2004; Twyman et al., 2003; Ma et al., 2003; Schillberg et al., 2003; Daniell et al. 2001; Giddings et al., 2000). Plant expression systems have advantages over other systems: production costs are relatively low and plants cells are not susceptible to contamination by human pathogens as can occur in mammalian expression systems. Human collagens, human growth hormones and antibodies have been produced in plants and these plant-derived proteins appear to have biological activities similar to those of the native proteins. For example, recombinant antibodies produced in tobacco plants have the same sensitivity, specificity, and importantly, the same affinity as monoclonal antibodies produced by the original hybridoma cell line (Voss et al., 1995).

Using transgenic plants for recombinant protein production has the drawback of resulting in generally low yields of the protein of interest. For some bacterial, animal and human proteins expressed in plant systems, yields vary widely and can be as low as 0.0001% TSP. Generally the greatest problems are encountered when there is a large evolutionary distance between the donor organism (the organism from which the gene of interest has been isolated) and the host organism (the plant host used to express the gene of interest). For example, in the field of edible vaccines, attempts are made to express a microbial protein (the antigen) in edible parts of transgenic plants (eg. maize, tomato and potato). Thus, one of the key challenges in the area of molecular pharming/farming is the employment of viable strategies to enhance expression levels and to improve the stability of the protein of interest (reviewed in Schillberg et al., 2003; Fischer et al., 2004; Stoger et al., 2005). This must be addressed in order to make plant-based systems useful and truly economical for the production of recombinant proteins (Hood, 2004). To date, several strategies have been used to attempt to achieve this (Schillberg et al., 2003; Fischer et al., 2004; Stoger et al., 2005).

Mucopolysaccharidosis (MPS) I is a lysosomal storage disease characterized by the deficiency of α-L-iduronidase, an enzyme involved in the stepwise degradation of glycosaminoglycans; in severely affected humans this genetic disease leads to death in early childhood because of profound skeletal, cardiac and neurological disturbances (Scott et al., 1995; Neufeld and Meunzer, 2001). Lysosomal storage diseases (that collectively represent over 50 disorders) are generally amenable to enzyme therapies (ERT or Enzyme Replacement Therapy) (reviewed in Brady, 2003; Desnick and Schuchman, 2002; Sly, 2000).

The plant B3 domain transcription factor ABI3 (ABscisic acid Insensitive3) plays an important role in the regulation of ABA responsive genes in developing seeds, particularly those required for reserve deposition, dormancy inception, and the acquisition of desiccation tolerance (reviewed in Bonetta and McCourt 1998; Finkelstein et al., 2002; Giraudat et al., 1994; Kermode and Finch-Savage, 2002; Koornneef et al., 2002; McCarty, 1995; Rohde et al., 2000). In mutants in which ABI3/VP1 genes are defective, the mutants seeds are not only disrupted in developmental processes but often also exhibit an altered or premature activation of post-germinative gene expression (Paek et al., 1998; Suzuki et al., 2001). Ectopically expressed ABI3 protein (effected by stable transformation of Arabidopsis with a chimeric 35S-ABI3 gene) leads to the re-activated expression of seed-specific genes in vegetative tissues and seedlings (Parcy and Giraudat, 1997; Parcy et al., 1994). There is a functional conservation among different ABI3/VP1 homologues (orthologues) as demonstrated by the successful complementation (rescue) of the severe Arabidopsis abi3 mutant (abi3-6) by transgenic expression of either the monocot VP1 gene (Suzuki et al., 2001) or the conifer CnABI3 gene (Zeng and Kermode, 2005). ABI3/VP1 proteins contain four conserved domains: an acidic activation domain and three basic domains, B1, B2 and B3 (Giraudat et al., 1992; McCarty et al., 1991). ABI3 is thought to regulate seed storage-protein gene expression by acting synergistically with other transcription factors (e.g. FUS3 and LEC1, LEC2 and others) that participate in combinatorial control (Kroj et al., 2003; Parcy et al., 1997; Finkelstein et al., 2002; Soderman et al., 2000; Nambara et al., 2000). ABI3/VP1 may recruit additional DNA-binding proteins to the promoters of storage-protein genes via its ability to alter chromatin structure (e.g. nucleosome positioning) (Li et al., 2001). Regulation of the expression of an Arabidopsis 2S storage protein gene (At2S3) appears to involve FUS3 and LEC2 that bind directly to promoter elements (RY repeats 1 and 2), while ABI3 acts in an indirect manner (likely via its interaction with bZIP proteins that bind to the G-box) (Kroj et al., 2003). ABI5 (a bZIP transcription factor) interacts directly via the B1 domain of ABI3 and two of the conserved charged domains of ABI5 that contain putative phosphorylation residues (Nakamura et al., 2001). ABI5 binding to ABREs (ABA Responsive Elements) may tether ABI3 to target promoters and facilitate the interaction of ABI3 with RY elements (a consensus sequence conserved in many seed-specific gene promoters) and transcription complexes (Finkelstein et al., 2002). The B2 domain of ABI3 is required for ABA-regulated gene expression and appears to facilitate the DNA binding capacity of a number of diverse DNA binding proteins (Carson et al., 1997; Hill et al., 1996). Moreover, interactions between the B2 and B3 domains, can mediate activation of target genes by interacting with different cis-acting DNA elements on those genes (Ezcurra et al., 2000).

### SUMMARY OF THE INVENTION

In various aspects, the present invention provides methods to enhance the expression of human/animal/plant proteins in transgenic plant cells, plants or plant tissues. In one embodiment, the invention provides an expression cassette for synthesis of the recombinant protein of interest. This cassette uses the cDNA encoding the mature plant/animal/human protein flanked by regulatory sequences (the promoter, 5′ untranslated region, signal peptide and one polyadenylation region—the 3′ untranslated region). In one embodiment, these sequences are derived from the arcelin gene. The construct may be represented as P-5′-UTR-SP-X-3′-UTR, wherein P is an ABA/ABI3-responsive promoter (or promoters in which ABA/ABI3-responsive elements are added) and X is a lysosomal enzyme or other human/animal/plant protein to be expressed in plant cells. Other regions (5′-UTR, SP and 3′ end) may for example be derived from other plant genes including (but not restricted to) a LEA, storage-protein or arcelin gene. In alternative embodiments, the 5′UTR could include a plant viral omega sequence. In the present example using human iduronidase as the target human protein, these various regions/sequences come from the arcelin gene, and surprising levels of expression are illustrated with particular constructs. If the protein of interest should undergo transport through the endomembrane system (eg. certain glycoproteins) a plant secretion signal peptide may be included. Similarly, a carboxy-terminal SEKDEL sequence for retention of the recombinant protein in the plant ER may be added, but is optional. The recombinant proteins are not limited to lysosomal enzymes, nor are they limited to glycoproteins. A wide range of proteins can be expressed in plant cells in this manner such as vaccines, antibodies, growth factors, hormone peptides, anticoagulants, nutritional supplements and the like.

The efficacy of the invention, as it pertains to the use of plants to generate recombinant proteins, is demonstrated by the generation of stably transformed tobacco plants co-expressing human α-L-iduronidase and an ABI3 gene ortholog of yellow-cedar (Chamaecyparis nootkatensis). Co-expression of the ABI3 gene may be achieved by the use of a constitutive promoter (eg. 35S CaMV), or by a leaf-specific, root-specific, tuber-specific, or even seed-specific promoter, depending upon the plant tissue hosting expression of the foreign protein of interest. In the present example, the human α-L-iduronidase (IDUA) can be purified (Clements et al., 1985, 1989; Downing et al., 2006) and further processed in vivo or in vitro to a specialized (e.g. phosphorylated) form for research or therapeutic uses.

The invention also includes but is not limited to the following modifications: (a) addition of regulatory DNA sequences (the 5′ promoter sequences, 5′ UTR, and 3′ UTR) and a signal peptide-encoding region from other genes, i.e., not just the arcelin gene; (b) addition of coding sequences or mRNA localization sequences (Crofts, et al. 2004; Choi et al., 2000) to direct the targeting of the recombinant protein to ER-derived protein bodies or another Golgi-independent transport destination (e.g. Jiang and Sun, 2002). If additional (non-native) amino acids have been added, they can later be cleaved in vivo or in vitro to produce the final proteins. (c) The expression system may include plant mutants that are deficient in N-acetylglucosamine transferase I (Von Schaewen et al., 1993; Gomez and Chrispeels, 1994) to control the maturation of N-linked glycans on the recombinant protein of interest (Zhao et al., 1997; Gomord and Faye, 2004). This encompasses the processes associated with complex glycan formation, including the addition of xylose and/or fucose sugar residues that have been shown to be immunogenic and to greatly reduce the efficacy of plant-derived recombinant proteins for pharmaceutical or other uses (Bardor et al., 2003).

The strategies described herein are not limited to expression of recombinant proteins in tobacco and, with appropriate changes to promoter and other sequences (and to the specific ABI3/VP1 orthologue used for co-expression), can be extended to include seeds, cultured cells, and vegetative tissues of any other plant species. Changes to the culture conditions during incubation treatments could also exploit the synergism between ABA and other hormones and between ABA and sugars (Finkelstein et al., 2002). They could also make use of stress treatments that lead to enhanced endogenous ABA levels or signaling. Up-regulation of proteins that interact with ABI3/VP1 to transactivate target promoters (including, but not restricted to ABI4/5, FUS and LEC transcription factors) or other proteins that otherwise regulate ABI3 (ABI3/VP1-interacting proteins and CnAIPs) (Jones et al., 2000; Kurup et al., 2000) may also be exploited in the technology.

### BRIEF DESCRIPTION OF THE DRAWINGS AND TABLE

FIG. 1. IDUA expression in transgenic Arabidopsis wild-type (WT) seeds and in Arabidopsis cgl mutant seeds. The Arabidopsis cgl mutant is deficient in the activity of N-acetylglucosaminyl transferase I (EC 2.4.1.101), the first enzyme in the pathway of complex glycan biosynthesis; this mutant avoids maturation of the N-linked glycans of IDUA (Downing et al., 2006). (a) Schematic diagram of ARC5s3, the gene construct used to express IDUA in Arabidopsis seeds, showing the 5′ flanking region (which includes the 5′ UTR), 3′ flanking region and signal-peptide encoding sequences (s), all derived from the ARC5-I gene, and the human IDUA mature coding region (hIDUA). (b) Western blot of soluble protein extracts from seeds of independent transformed WT lines (lanes 2-8). UT=untransformed WT seeds (far left lane). Equal amounts of protein were loaded (100 μg). Numbers indicate the molecular weights (kDa) of the size markers (MW) and the immunoreactive IDUA-related polypeptides. (c) IDUA activities of soluble extracts from seeds of 29 independent transformed lines. UT=untransformed WT seeds. One unit is defined as 1 nmol 4 MU/min. (d) Western blot of soluble protein extracts from seeds of independent transformed cgl lines (lanes 2-8). cgl=untransformed cgl seeds (far left lane). Lane 9 (1*) is the highest-expressing transgenic WT line (i.e. line 1 of FIGS. 1b and 1c). Numbers indicate the molecular weights of the size markers (MW) and the immunoreactive IDUA-related polypeptides. (e) IDUA activities of soluble extracts from seeds of 29 independent transformed cgl lines. cgl=untransformed cgl seeds. IDUA activity and protein levels are significantly higher in transgenic cgl versus wild-type seeds. (f) Shows αL-iduronidase activities of three atypical ARC5s3 lines (cgl background) with extremely high levels of α-L-iduronidase gene expression.

FIG. 2. A. Schematic diagram of constructs for testing the expression of the gene encoding the human lysosomal enzyme, α-L-iduronidase, in Arabidopsis cgl mutant seeds. Gene constructs differ in 5′-UTR-signal peptide sequences, and in 3′-UTR-flanking sequences. B. Table of α-L-iduronidase activities (units per mg TSP) and α-L-iduronidase protein in extracts of the highest-expressing transformed lines determined from the screening of at least 30 independent transgenic lines for each construct. The table also shows α-L-iduronidase activities of three atypical ARC5s3 lines with extremely high levels of α-L-iduronidase gene expression. One unit is defined as 1 nmol 4 MU/min.

Table 1. Specific activities of Arabidopsis-derived α-L-iduronidase following purification of the recombinant enzyme from T₃ seeds using a modified three-column procedure developed for extraction from human liver (Clements et al., 1989). The specific activity of the enzyme following chromatography on Bio-Gel P-100 was 14,700 nmol 4 MU/min/mg TSP, comparable to that of the enzyme isolated from several mammalian sources (Kakkis et al., 1994; Ohshita et al., 1989, Schuchman et al., 1984). The overall recovery from transformed WT and cgl seeds is summarized in Table 1. The results illustrate that plant-produced human IDUA displays specific activity comparable to that of mammalian systems.

FIG. 3. Gene constructs for co-expression in transgenic tobacco. The examples show one construct for the synthesis of the bacterial reporter protein GUS (Vic-GUS; construct b) and two constructs for synthesis of the human lysosomal enzyme α-L-iduronidase (Arc-hIDUA and Arc-hIDUA-KDEL; constructs c and d). The final construct (construct a) is one for the ectopic expression of a plant (yellow-cedar) ABI3 gene. Co-expression of construct (a) encoding the transcription factor ABI3 and either of constructs (b), (c) or d) causes the “ectopic” activation of the chimeric (GUS or iduronidase) genes driven by the seed gene promoters (vicilin and arcelin promoters, respectively). This allows for high-level expression of the recombinant proteins (bacterial GUS and human iduronidase) in the vegetative tissues of transgenic tobacco. Transformants expressing constructs (b, (c) or (d) alone serve as controls for comparison.

FIG. 4. Effect of natural S-(+)-ABA on recombinant bacterial β-glucuronidase (GUS) activities in transgenic tobacco leaves co-expressing construct (a) (the CnABI3 gene) and construct (b) (encoding GUS). In the presence of natural S-(+)-ABA, the CnABI3 protein transactivates the vicilin promoter and this leads to enhanced GUS activities. There is a greater enhancement of GUS activities, with an increasing concentration of natural ABA up to 200 μM.

FIG. 5. Enhancement of recombinant human α-L-iduronidase activities in transgenic tobacco in the presence of the ABI3 protein. Transgenic tobacco leaves expressing constructs c or d alone (Arc or AK, black bars) have very little α-L-iduronidase activity. However, in the presence of the ABI3 protein (i.e. in tobacco leaves co-expressing constructs a and c or constructs a and d; Arc & ABI3 [upper figure, gray bars] or AK & ABI3 [lower figure, gray bars]), there is major increase in the yield (activity) of the recombinant protein. Wt=non-transformed tobacco leaves.

FIG. 6. Use of ABA to enhance human α-L-iduronidase activity in plants co-expressing ABI3 and α-L-iduronidase. When leaves of selected tobacco co-transformants (plants co-expressing constructs a and d [Arc-IDUA-KDEL/ABI3]) are incubated in natural ABA (S(+)-ABA at 80 μM), there is a further enhancement of α-L-iduronidase activity levels. For example, at day 7 of incubation, in comparison to the transgenic control leaves (leaves placed in culture media containing no ABA), ABA enhances the activity of α-L-iduronidase by ˜58-fold.

FIG. 7. Effects of different concentrations of ABA on the enhancement of human α-L-iduronidase activities in plants co-expressing ABI3 and α-L-iduronidase. When leaves of selected tobacco co-transformants (plants co-expressing constructs a and c [Arc-IDUA/ABI3] and plants co-expressing constructs a and d [Arc-IDUA-KDEL/ABI3]) are incubated for 6 days in increasing concentrations of S(+)-ABA, the α-L-iduronidase activities increase, reaching a maximum at 150 μM S(+)-ABA.

FIG. 8. ABA acts at the transcriptional level to enhance the levels of human α-L-iduronidase. Shows Northern blot analysis of tobacco leaves co-expressing constructs a and c [Arc-IDUA/ABI3] or co-expressing constructs a and d [Arc-IDUA-KDEL/ABI3]) incubated on culture medium containing 100 μM S(+)-ABA (or no ABA, C), for 6 days. When ABA is present, the leaves show enhanced steady-state mRNA levels encoding α-L-iduronidase as compared to transgenic control leaves (leaves placed in culture without ABA). Analog-1 (a chemically modified ABA molecule) is added as a positive control, again showing the positive action of ABA on recombinant gene/protein expression.

FIG. 9. Western blot showing the effects of ABA at different concentrations of the levels of human α-L-iduronidase protein at day 6 of incubation. As with the activity data (FIG. 7), leaves of selected tobacco co-transformants (plants co-expressing constructs a and c [Arc-IDUA/ABI3] and plants co-expressing constructs a and d [Arc-IDUA-KDEL/ABI3]) incubated for 6 days in increasing concentrations of S(+)-ABA, show the maximum α-L-iduronidase protein accumulation levels at 150 μM S(+)-ABA. Analog-1 (a chemically modified ABA molecule) is added as a positive control again showing the positive action of ABA on recombinant protein accumulation.

FIG. 10. The effects of different treatments designed to enhance endogenous ABA levels on human α-L-iduronidase activities. Leaves of selected tobacco co-transformants (plants co-expressing constructs a and c [Arc-IDUA/ABI3] and plants co-expressing constructs a and d [Arc-IDUA-KDEL/ABI3]) were incubated for 6 days in media containing the following chemicals: (1) polyethylene glycol, PEG; (2) mM NaCl; (3) mM sucrose; (4) mM mannitol, or the leaves were kept on medium at 4 degrees celsius. As compared to the ABA control (150 μM S(+)-ABA), the higher concentration NaCl treatments (240 mM or 300 mM) show dramatic effectiveness in enhancing α-L-iduronidase activities.

FIG. 11 illustrates various B3 DNA Binding Domains, which may be utilized in alternative promoters of the invention, such as ABA responsive promoters.

FIG. 12 illustrates the Arabidopsis thaliana ABI3 protein sequence from GenBank Accession NP_{—}189108 (see Giraudat, J., Hauge, B. M., Valon, C., Smalle, J., Parcy, F. and Goodman, H. M., Isolation of the Arabidopsis ABI3 gene by positional cloning. Plant Cell 4 (10), 1251-1261 (1992).

FIG. 13A to 13H illustrates BLAST sequence comparisons between A. thaliana ABI3 and various homologous sequences, illustrating alternative transcription factor sequences of the invention, for example having sequences corresponding to regions of homology illustrated in the Figure.

### DETAILED DESCRIPTION OF THE INVENTION

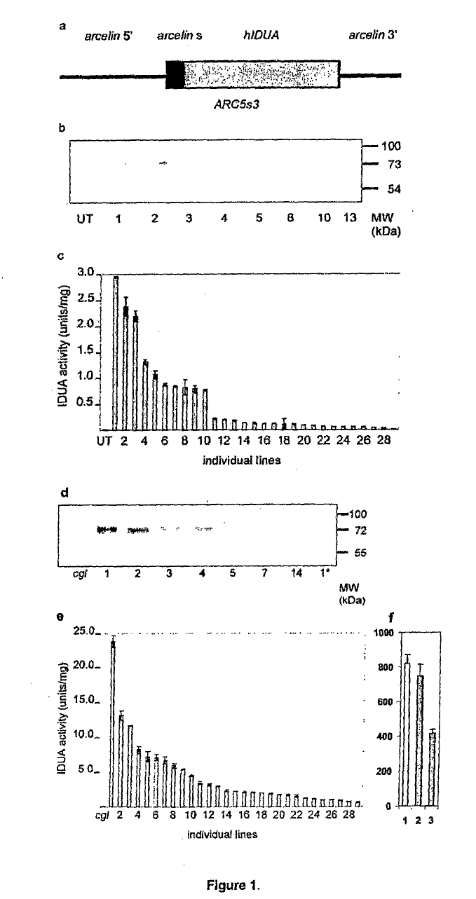

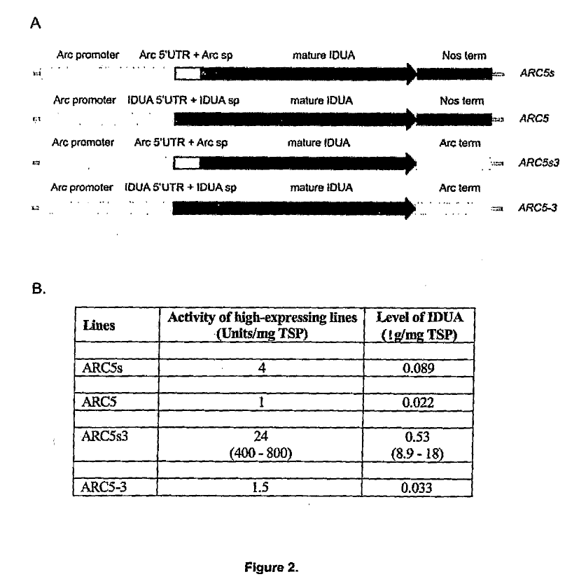

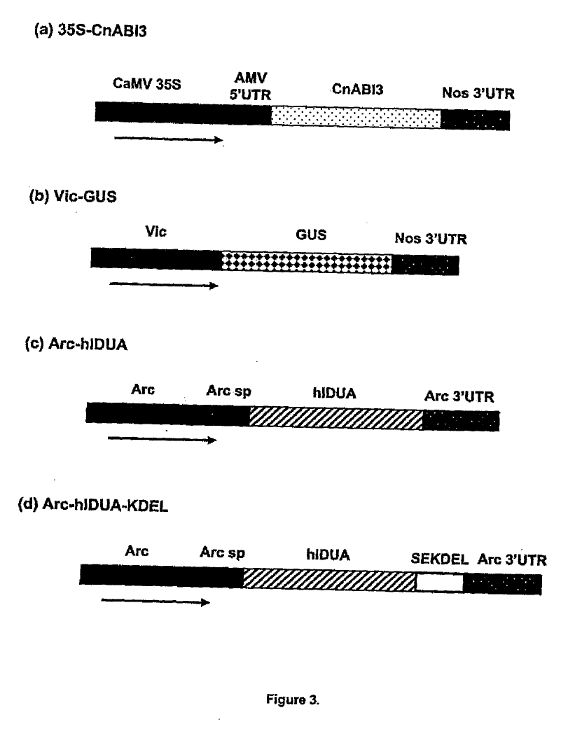

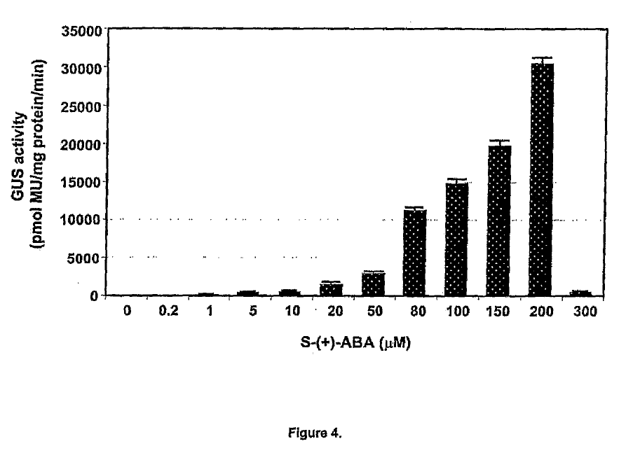

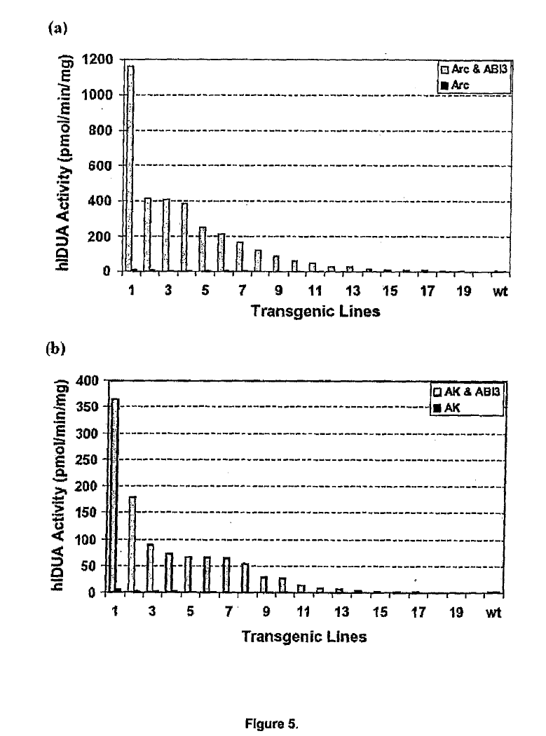

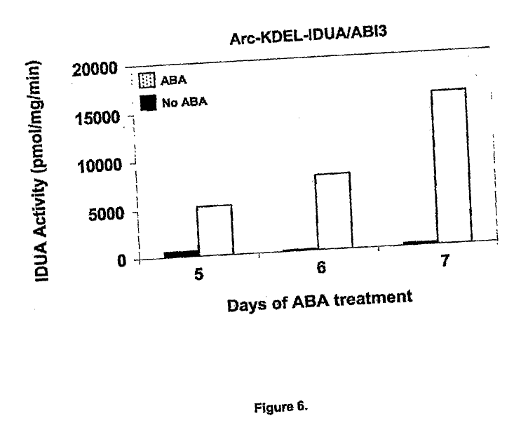

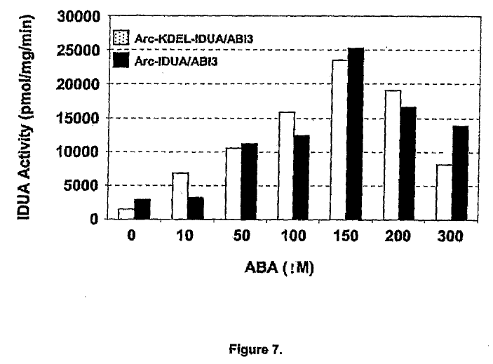

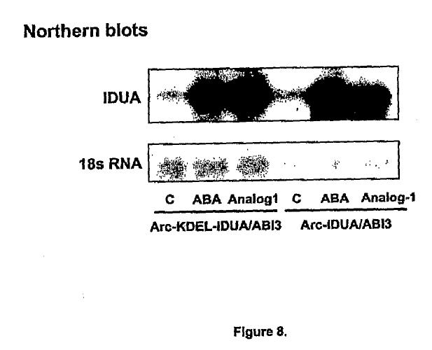

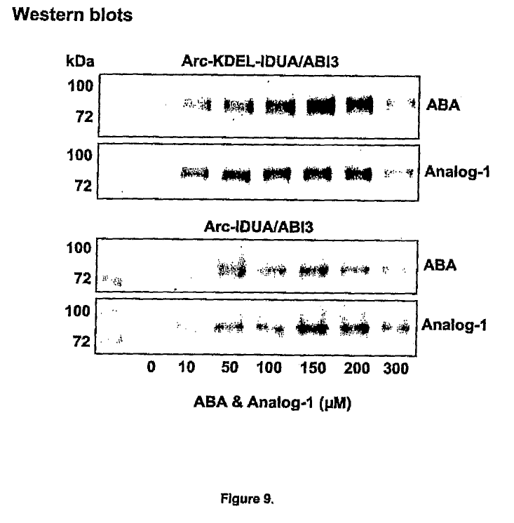

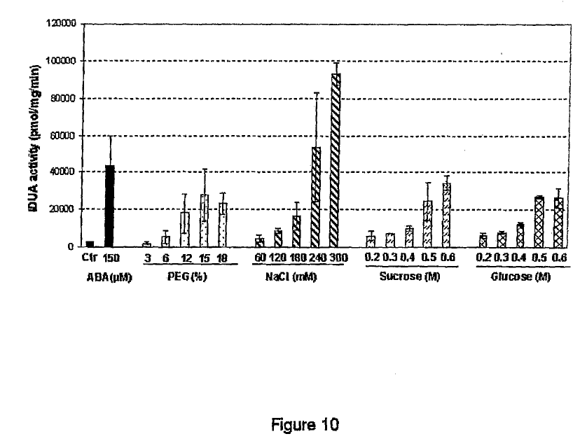

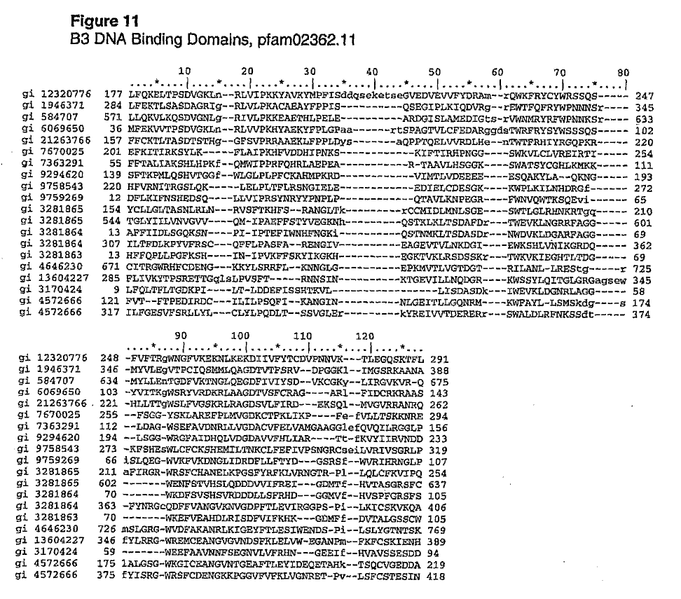

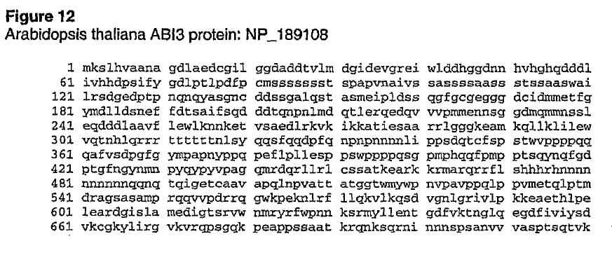

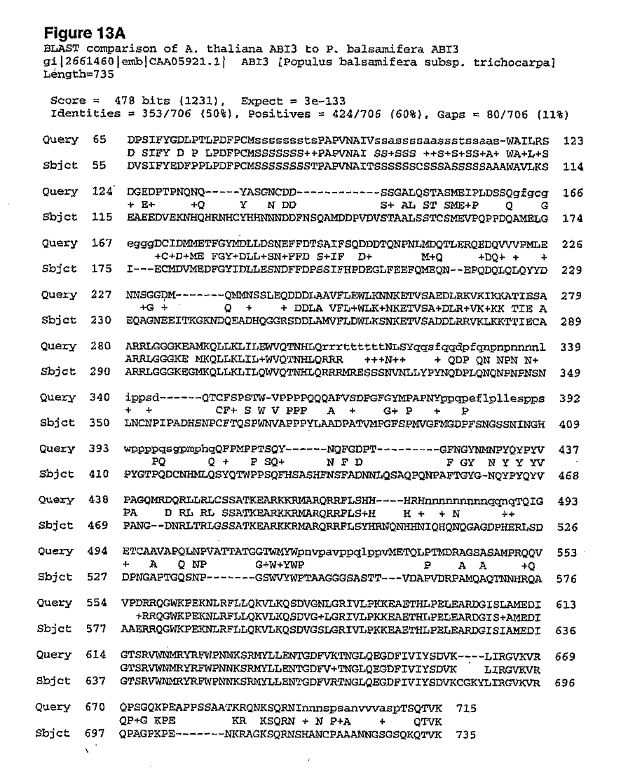

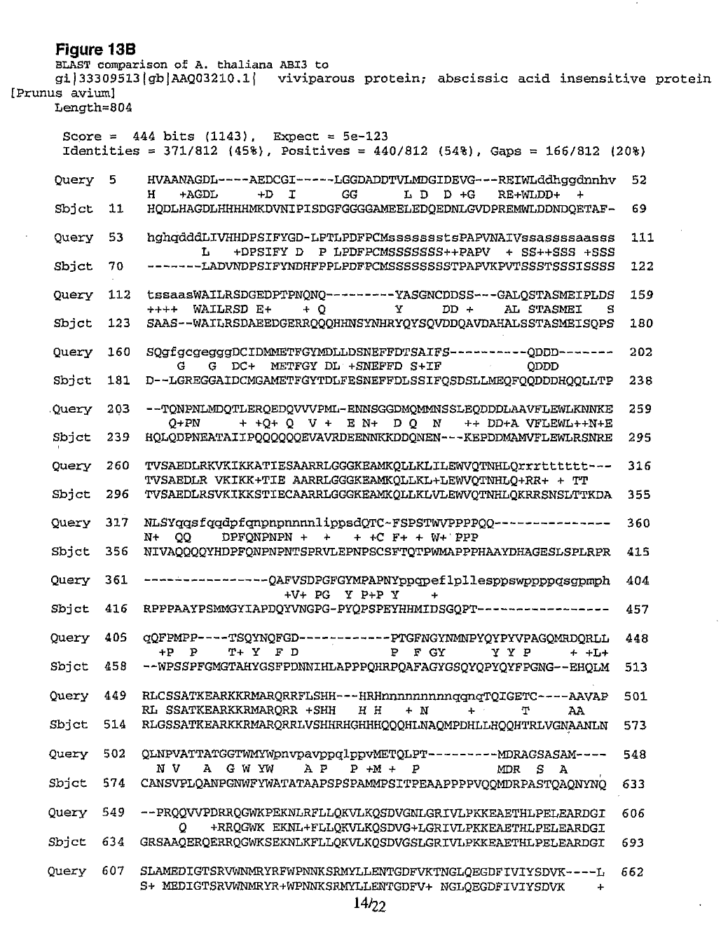

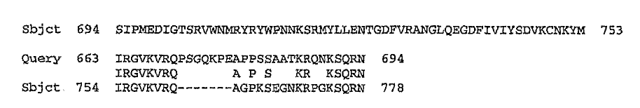

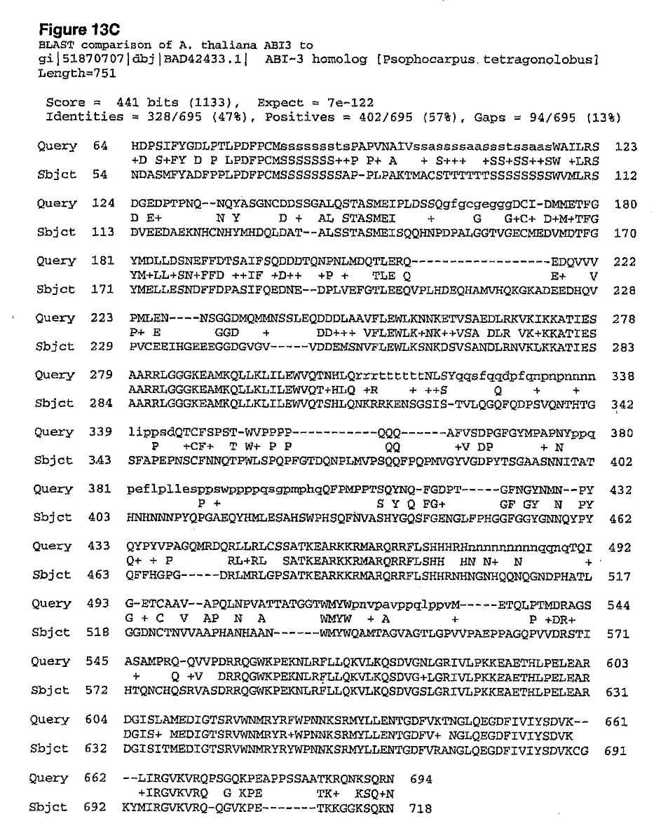

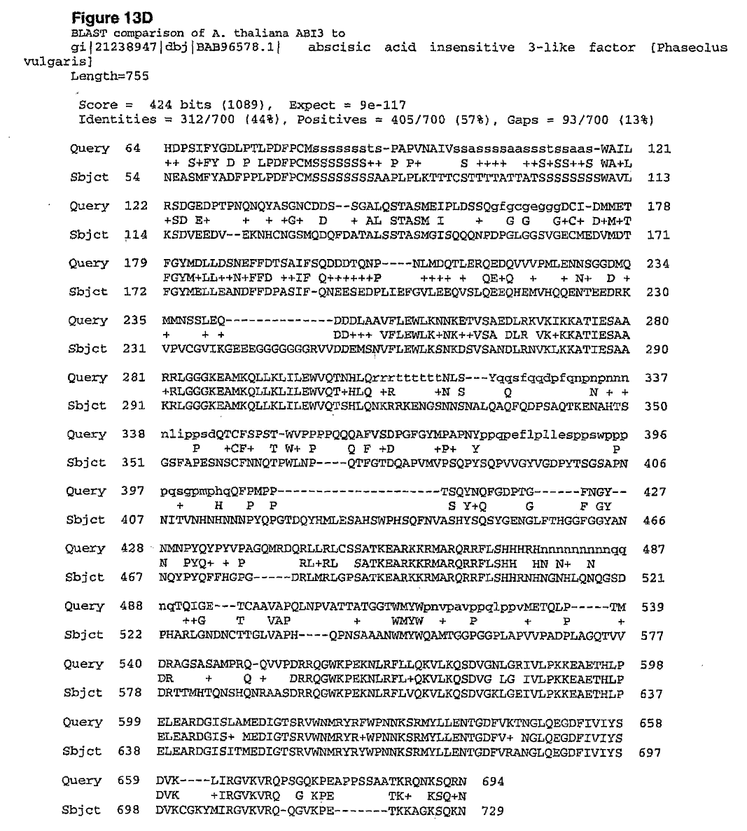

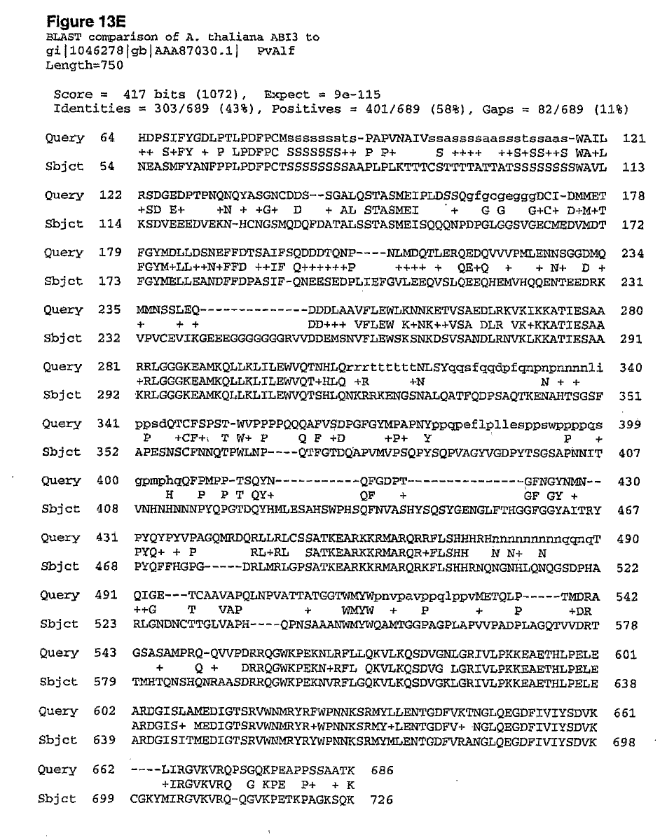

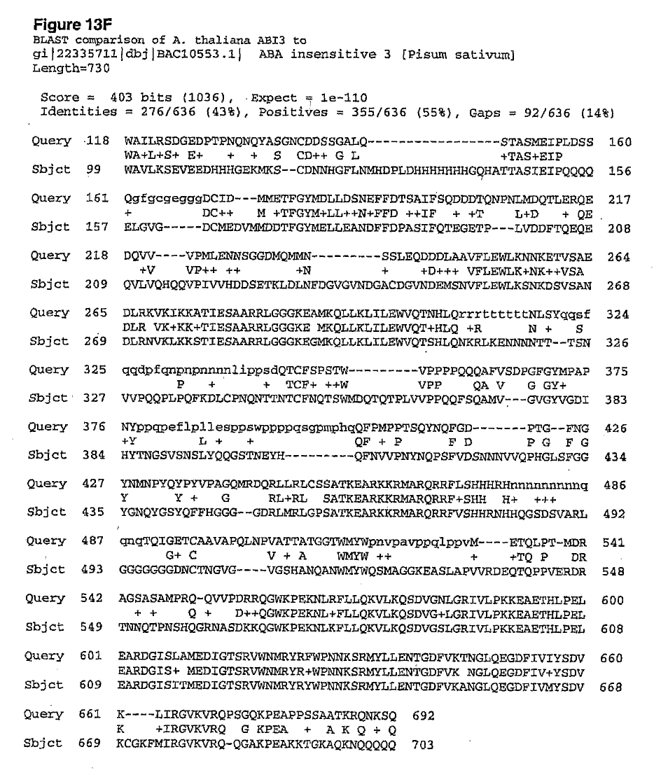

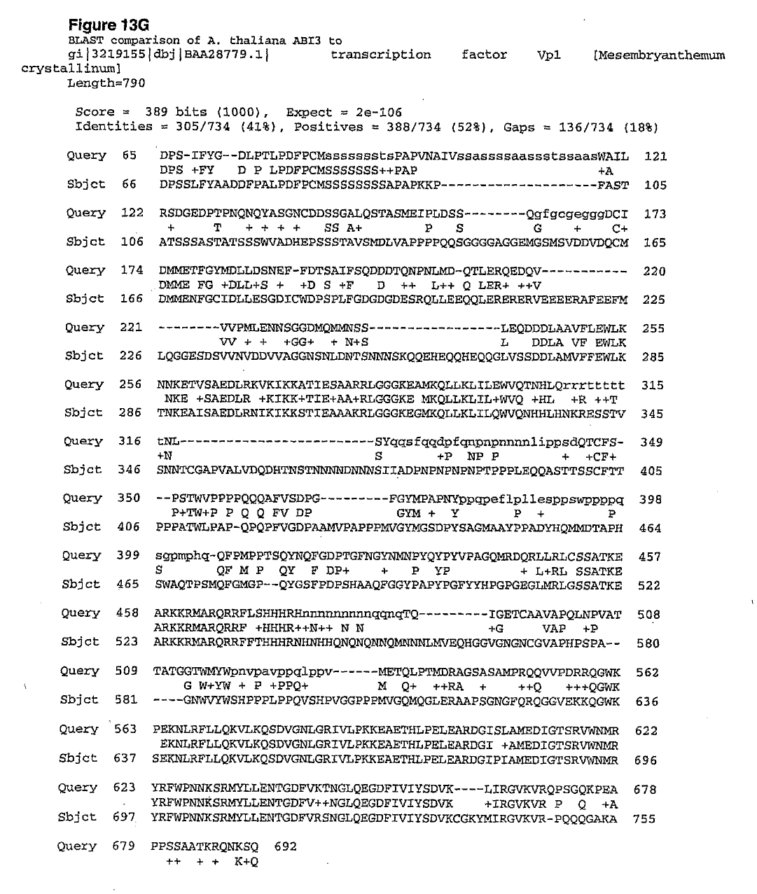

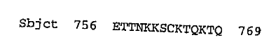

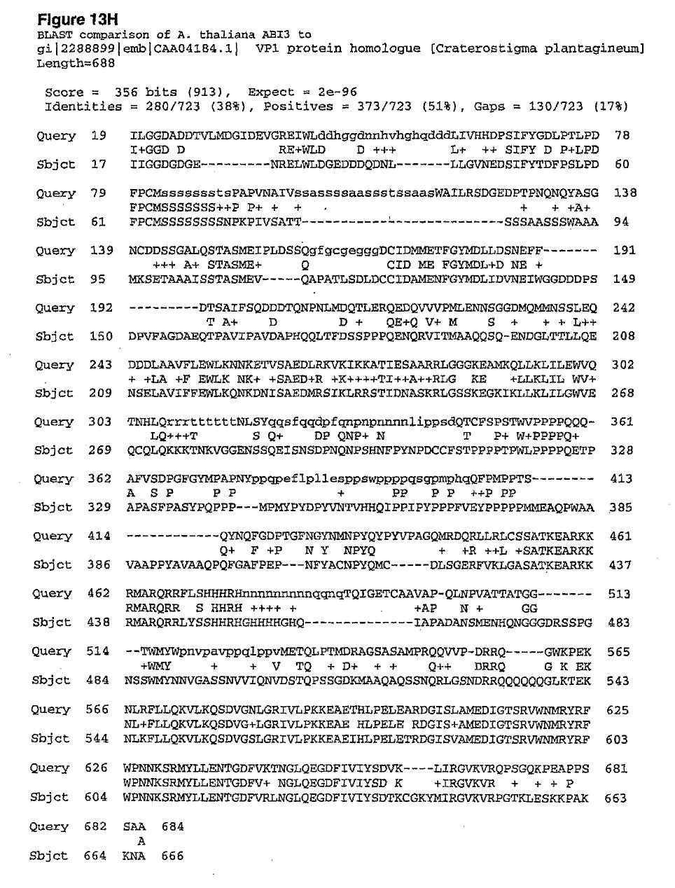

The invention is in the field of production of recombinant proteins. Specifically this invention relates to enhancing the yield of recombinant human, plant and animal protein (lysosomal proteins, hormone peptides, anticoagulants, growth factors, enzymes, defensive proteins, storage proteins and the like) in a plant system. The constructs for co-expression in selected embodiments are shown in FIG. 1, which includes one construct for the synthesis of the human lysosomal enzyme α-L-iduronidase and a second construct for ectopic expression of a plant (yellow-cedar) ABI3 gene. The principle of the technology is demonstrated by expressing a recombinant protein of interest in which the cDNA encoding the mature animal/human/plant protein is flanked by regulatory sequences (the promoter, 5′ untranslated region, signal peptide and 3′ untranslated region) of the Phaseolus vulgaris arcelin gene. A carboxy-terminal SEKDEL sequence for retention of the recombinant protein in the plant ER is optional. Although the arcelin promoter is generally seed-specific, chimeric genes driven by this and other promoters (e.g. seed storage protein gene promoters) can be ectopically activated in plant vegetative tissues in the presence of the transcription factor ABI3 (ABscisic acid Insensitive3). Herein we show that the constitutive synthesis of the ABI3 transcription factor leads to a transactivation of the arcelin promoter and accordingly higher activity and levels of a human recombinant protein (α-L-iduronidase) result, particularly in the presence of the phytohormone ABA. The invention provides the means of enhancing the yields of recombinant proteins in transgenic plants (both vegetative tissues and seeds). The invention is demonstrated by a working example in which transgenic tobacco leaves co-express genes encoding the human lysosomal enzyme α-L-iduronidase and an ABI3 gene of yellow-cedar (Chamaecyparis nootkatensis).

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be at least 10%, 20%, 30%, 40%, 50%, 52.5%, 55% or 60% or 75%, or more generally at least 80%, 85%, 90%, or 95%, or as much as 99% or 100% identical at the amino acid or nucleotide level to the sequence used for comparison using, for example, the Align Program (Myers and Miller, CABIOS, 1989, 4:11-17) or FASTA. For polypeptides, the length of comparison sequences may be at least 4, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, insertions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

The terms “nucleic acid” or “nucleic acid molecule” encompass both RNA (plus and minus strands) and DNA, including cDNA, genomic DNA, and synthetic (e.g., chemically synthesized) DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. One example of a modified RNA included within this term is phosphorothioate RNA. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). Thus a “cDNA clone” means a duplex DNA sequence complementary to an RNA molecule of interest, carried in a cloning vector.

An “isolated nucleic acid” is a nucleic acid molecule that is free of the nucleic acid molecules that normally flank it in the genome or that is free of the organism in which it is normally found. Therefore, an “isolated” gene or nucleic acid molecule is in some cases intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). In some cases, an isolated nucleic acid molecule is intended to mean the genome of an organism such as a virus. An isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstances, the material may be purified to essential homogeneity, for example as determined by PAGE or column chromatography such as HPLC. The term therefore includes, e.g., a genome; a recombinant nucleic acid incorporated into a vector, such as an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant nucleic acid which is part of a hybrid gene encoding additional polypeptide sequences. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Recombinant DNA contained in a vector are included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules. Such isolated nucleic acid molecules are useful in the manufacture of the encoded polypeptide, as probes for isolating homologous sequences (e.g., from other species), for gene mapping (e.g., by in situ hybridization with chromosomes), or for detecting expression of the nucleic acid molecule in tissue (e.g., human tissue, such as peripheral blood), such as by Northern blot analysis.

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

As used herein, “heterologous” in reference to a nucleic acid or protein is a molecule that has been manipulated by human intervention so that it is located in a place other than the place in which it is naturally found. For example, a nucleic acid sequence from one species may be introduced into the genome of another species, or a nucleic acid sequence from one genomic locus may be moved to another genomic or extrachromasomal locus in the same species. A heterologous protein includes, for example, a protein expressed from a heterologous coding sequence or a protein expressed from a recombinant gene in a cell that would not naturally express the protein.

By “complementary” is meant that two nucleic acid molecules, e.g., DNA or RNA, contain a sufficient number of nucleotides that are capable of forming Watson-Crick base pairs to produce a region of double-strandedness between the two nucleic acids. Thus, adenine in one strand of DNA or RNA pairs with thymine in an opposing complementary DNA strand or with uracil in an opposing complementary RNA strand. It will be understood that each nucleotide in a nucleic acid molecule need not form a matched Watson-Crick base pair with a nucleotide in an opposing complementary strand to form a duplex.

By “vector” is meant a DNA molecule derived, e.g., from a plasmid, bacteriophage, or mammalian or insect virus, or artificial chromosome, that may be used to introduce a polypeptide, into a host cell by means of replication or expression of an operably linked heterologous nucleic acid molecule. By “operably linked” is meant that a nucleic acid molecule such as a gene and one or more regulatory sequences (e.g., promoters, ribosomal binding sites, terminators in prokaryotes; promoters, terminators, enhancers in eukaryotes; leader sequences, etc.) are connected in such a way as to permit the desired function e.g. gene expression when the appropriate molecules (e.g., transcriptional activator proteins) are bound to the regulatory sequences. A vector may contain one or more unique restriction sites and may be capable of autonomous replication in a defined host or vehicle organism such that the cloned sequence is reproducible. By “DNA expression vector” is meant any autonomous element capable of directing the synthesis of a recombinant peptide. Such DNA expression vectors include bacterial plasmids and phages and mammalian and insect plasmids and viruses. A “shuttle vector” is understood as meaning a vector which can be propagated in at least two different cell types, or organisms, for example vectors which are first propagated or replicated in prokaryotes in order for, for example, subsequent transfection into eukaryotic cells. A “replicon” is a unit that is capable of autonomous replication in a cell and may includes plasmids, chromosomes (e.g., mini-chromosomes), cosmids, viruses, etc. A replicon may be a vector.

A “host cell” is any cell, including a prokaryotic or eukaryotic cell, into which a replicon, such as a vector, has been introduced by for example transformation, transfection, or infection.

An “open reading frame” or “ORF” is a nucleic acid sequence that encodes a polypeptide. An ORF may include a coding sequence having i.e., a sequence that is capable of being transcribed into mRNA and/or translated into a protein when combined with the appropriate regulatory sequences. In general, a coding sequence includes a 5′ translation start codon and a 3′ translation stop codon.

A “transcriptional regulatory sequence” “TRS” or “intergenic sequence” is a nucleotide sequence that lies upstream of an open reading frame (ORF) and serves as a template for the reassociation of a nascent RNA strand-polymerase complex.

A “peptide,” “protein,” “polyprotein” or “polypeptide” is any chain of two or more amino acids, including naturally occurring or non-naturally occurring amino acids or amino acid analogues, regardless of post-translational modification (e.g., glycosylation or phosphorylation). An “polyprotein”, “polypeptide”, “peptide” or “protein” of the invention may include peptides or proteins that have abnormal linkages, cross links and end caps, non-peptidyl bonds or alternative modifying groups. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain. Alternatively, the modifying group can be coupled to a side chain of at least one amino acid residue of a peptidic structure, or to a peptidic or peptido-mimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

A “signal sequence” or “signal peptide” is a sequence of amino acids that may be identified, for example by homology or biological activity to a peptide sequence with the known function of targeting a polypeptide to a particular region of the cell. A signal sequence or signal peptide may be a peptide of any length, that is capable of targeting a polypeptide to a particular region of the cell. In some embodiments, the signal sequence may direct the polypeptide to the cellular membrane so that the polypeptide may be secreted, a “secretion signal sequence” or “secretion signal peptide”. In alternate embodiments, the signal sequence may direct the polypeptide to an intracellular compartment or organelle, such as the ER. In alternate embodiments, a signal sequence may range from about 13 or 15 amino acids in length to about 60 amino acids in length. Secretion signal sequences are for example disclosed in the following documents: Choo K H, Tan T W, Ranganathan S. 2005. SPdb—a signal peptide database. BMC Bioinformatics 6:249; Nothwehr, S. F. and J. I. Gordon. 1989; Eukaryotic signal peptide structure/function relationships. Identification of conformational features which influence the site and efficiency of co-translational proteolytic processing by site-directed mutagenesis of human pre(delta pro)apolipoprotein A-II. J Biol Chem 264: 3979-3987; and, McGeoch, D. J. 1985. On the predictive recognition of signal peptide sequences. Virus Res 3: 271-286.

In various embodiments of the invention, an ABI3 transcription factor is used. In one aspect of the invention, ABI3 transcription factors may include derived peptides that differ from a portion of a native ABI3 sequence by conservative amino acid substitutions. As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. In some embodiments, for example an ABI3 transcription factor may be a transcription factor comprising a B3 DNA-binding domain (which binds to an RY motif CATGCA(TG)) and at least one transcription activation domain. In some embodiments, the ABI3 transcription factor may be a naturally occurring ABI3 transcription factor, or a recombinant ABI3 transcription factor that has a high degree of homology to a naturally occurring ABI3 transcription factor sequence.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (O); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-naphthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (J. Mol. Bio. 179:125-142, 184). Genetically encoded hydrophobic amino acids include Gly, Ala, Phe, Val, Leu, Ile, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Tryp, while non-genetically encoded aromatic amino acids include phenylglycine, 2-naphthylalanine, beta-2-thienylalanine, 1,2,3,4-tetrahydro-isoquinoline-3-carboxylic acid, 4-chlorophenylalanine, 2-fluorophenylalanine-3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids ornithine, 2,3-diaminopropionic acid, 2,4-diaminobutyric acid, and homoarginine.

It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

In various embodiments, the invention involves the use of 3′ untranslated regulatory sequences. Such sequence may for example be derived from native plant genes, such as seed specific protein genes, such as an arcelin gene, a vicilin gene or a napin gene. These sequences may for example comprise one or more of a polyadenylation signal, a downstream (G)T-rich sequence, a matrix attachment region (see for example THE PLANT CELL, Vol 1, Issue 7 671-680, Different 3′ End Regions Strongly Influence the Level of Gene Expression in Plant Cells. I L W. Ingelbrecht, L M F. Herman, R. A. Dekeyser, M. C. Van Montagu and A. G. Depicker; George C. Allen, Steven Spiker, William F. Thompson, Use of matrix attachment regions (MARs) to minimize transgene silencing, Plant Molecular Biology, Volume 43, Issue 2-3, June 2000, Pages 361-376; I. Liebich, J. Bode, I. Reuter and E. Wingender, Nucleic Acids Research, 2002, Vol. 30, No. 15 3433-3442, Evaluation of sequence motifs found in scaffold/matrix-attached regions).

In various embodiments, the invention utilizes promoter sequences, such as arcelin, vicilin or napin gene promoter sequences. U.S. Pat. No. 6,927,321 issued 9 Aug. 2005 describes arcelin promoters, and variants thereof. Alternative arcelin promoter sequences are also described in Osborn, et al. Science, 240:207-210, 1988), —2 (John, et al., Gene 86:171-176, 1990), —3, or—4 (Mirkov, et al., Plant Mol. Biol., 26:1103-1113, 1994) promoter. In the present application, an arcelin promoter is . . . a region that mediates transcription of an arcelin coding sequence in a naturally occurring arcelin gene. An arcelin coding sequence is a coding sequence that is functionally and structurally homologous to other arcelin coding sequences, such as the Phaseolus vulgaris mRNA sequences for arcelins: the arc3-I gene, GenBank Accession No. AJ534654; arc4-I gene, GenBank Accession Nos. AJ439716 or U10351. Arcelin coding sequences of the invention include sequences that encode proteins that are functionally and structurally homologous to other arcelin proteins, such as the arcelin protein of Phaseolus vulgaris, GenBank Accession CAD58972 (Lioi, L., Sparvoli, F., Galasso, I., Lanave, C. and Bollini, R., Lectin-related resistance factors against bruchids evolved through a number of duplication events. Theor. Appl. Genet. 107 (5), 814-822 (2003)). Vicilin gene promoter sequences may for example be sequences that are homologous to the Arabidopsis thaliana vicilin gene promoter (sequences of the A. thaliana gene are for example disclosed in GenBank Accession No. NC 003071, or protein GenBank Accession No. NP 180416. Napin gene promoter sequences are for example disclosed in the following documents: Mats Ellerström, Kjell Stalberg, Inés Ezcurra, Lars Rask, Functional dissection of a napin gene promoter: identification of promoter elements required for embryo and endosperm-specific transcription, Plant Molecular Biology, Volume 32, Issue 6, December 1996, Pages 1019-1027; and, Mats L. ERICSON1, Eva MURÉN1, Hans-Olof GUSTAVSSON1, Lars-Göran JOSEFSSON1 and Lars RASK, Analysis of the promoter region of napin genes from Brassica napus demonstrates binding of nuclear protein in vitro to a conserved sequence motif, European Journal of Biochemistry, Volume 197 Page 741—May 1991. In some embodiments, the invention may utilize promoters comprising abscisic acid-responsive elements (ABREs), such as CACGTGGC or GTACGTGGCGC.

The invention will be more readily understood by references to the following examples, which illustrate various alternative embodiments of the invention.

### EXAMPLE 1

### Construction of Vectors for Plant Expression of Human IDUA

### General Approach and Principles

Gene constructs are shown in FIG. 3. The gene regulatory sequences used to demonstrate the technology were chosen because of their ability to generate high-level expression of the human recombinant protein α-L-iduronidase (IDUA) in Arabidopsis seeds (FIGS. 1 & 2; Table 1). The promoter used in the example (the arcelin gene promoter) is classed as generally seed-specific; thus, it is expected to yield little or no expression of the (α-L-iduronidase (IDUA) gene in the vegetative tissues of transgenic plants. In principle, the expression cassette designed for expression of the recombinant protein need not be from the arcelin gene, but could be one of most of the ABA/ABI3-responsive promoters (e.g. those of LEA- or LEA-like genes, storage-protein genes and the oleosin gene as well as others). The “ectopic” activation of the chimeric gene in plant vegetative tissues is achieved by expression of a gene encoding the transcription factor ABI3. The strategy involves producing transgenic plants co-expressing a 35S-ABI3-Nos construct (Construct a; FIG. 3) and either construct (c) (arcelin 5′-arcelin signal peptide-IDUA-arcelin 3′) or (d) (arcelin 5′-arcelin signal peptide-IDUA-SEKDEL-arcelin 3′). Transformants expressing constructs (c) and (d) alone served as controls for comparison (FIG. 3).

### Methods

A 1201-bp DNA fragment comprising the 5′ flanking region, 5′ UTR and signal peptide-encoding sequences were derived from the arcelin-5-I gene (GenBank accession number Z50202) that was isolated from the wild common bean (Phaseolus vulgaris L., genotype G02771) (Goossens et al., 1995, 1999; Downing et al., 2006). These sequences were cloned by PCR and fused to sequences encoding the mature human α-L-iduronidase protein (Scott et al., 1991; GenBank accession no. M74715) (i.e. the IDUA cDNA minus sequences encoding the signal peptide). The 3′ end of the hIDUA cDNA was then fused with a 905-bp fragment containing the ARC5-I gene transcription terminator and 3′ flanking region to create construct (c) in FIG. 3 (construct ARC5s3 in FIGS. 1 & 2). Construct (d) contained the same 5′ and 3′ regulatory sequences present in construct (c); however sequences encoding SEKDEL were fused to the 3′ end of the hIDUA-encoding sequences to create a carboxy-terminal ER retention signal on the plant-produced recombinant human protein.

To co-express the CnABI3 protein and the human protein constructs (c or d of FIG. 3) in transgenic tobacco leaves, a chimeric construct containing the CnABI3 gene coding region (GenBank accession number AJ131113; Lazarova et al. 2002) was generated (Construct a, FIG. 3) to yield constitutive synthesis of the CnABI3 protein throughout all tissues of the plant. This construct contained the following regulatory sequences: (1) a modified 35S cauliflower mosaic virus promoter containing a duplicated 400-bp enhancer element; (2) the 5′-untranslated region from the alfalfa mosaic virus RNA 4 (AMV) (Datla et al., 1993) and (3) the 3′ end of the nopaline synthase (nos) gene (Depicker et al., 1982). The construct is denoted 35S-CnABI3 in FIG. 3 and was generated as previously described in Zeng et al. (2003).

### EXAMPLE 2

### Stable Expression Studies in Transgenic Tobacco Leaves

Construct (c) (FIG. 3) was cloned into the binary vector pBI101 and transformed into Agrobacterium tumefaciens strain GV3101. Construct (d) (FIG. 3) was cloned into the binary vector, pRD400. The CnABI3 construct (construct a) was cloned into the HindIII and EcoRI sites of the binary vector, pCambia, and transferred into LBA4404 Agrobacterium tumefaciens strain via electroporation (Zeng et al. 2003).

Transgenic tobacco plants were also generated by co-expressing the CnABI3 gene (construct a) and a gene construct containing the bacterial GUS gene coding region linked to a seed storage protein gene promoter—the vicilin gene promoter (construct b of FIG. 3) (Jiang et al., 1995).

Stably transformed plants were cultured in magenta boxes at 25° C. and sub-cultured every 3 months. Healthy, fully expanded leaves from 4-week plants were used in the present study.

### Ectopic Co-Expression of a Transcription Factor Enhances Production of Human IDUA

FIG. 5 shows that the constitutive synthesis of the ABI3 transcription factor leads to a transactivation of the arcelin promoter and accordingly higher hIDUA activity levels result.

### EXAMPLE 3

### Effects of ABA on Recombinant Protein Production in Stably Transformed Tobacco Leaves

### The Phytohormone ABA has a Synergistic Effect on Enhancing Recombinant Bacterial GUS and Human α-Iduronidase Expression in the Presence of the ABI3 Transcription Factor

FIGS. 4-9 show that the enhancement of bacterial GUS and human IDUA expression is particularly strong in the presence of the phytohormone ABA. For example, in cotransformed leaves of transgenic tobacco expressing the ABI3 gene (construct a) and the IDUA-KDEL gene (construct d), ABA elicited a 58-fold increase in IDUA activities after 7 days of incubation (FIG. 6). This led to IDUA activities in leaves as high as 16,000 pmol min⁻¹ mg⁻¹. ABA causes its enhancing effects on human IDUA expression at the level of increasing steady-state levels of mRNAs (FIG. 8). This enhanced gene expression in the presence of ABA is accompanied by an increased amount of IDUA protein (FIG. 9) and IDUA activity (FIGS. 6 & 7). The ABA concentration that appears maximal in terms of enhancing IDUA is 150 μM (FIGS. 7 & 9).

Human α-Iduronidase is Readily Purified from Transgenic Tissues

Table 1 shows the specific activities of Arabidopsis-derived α-L-iduronidase following purification of the recombinant enzyme from T₃ seeds using a modified three-column procedure developed for extraction from human liver (Clements et al., 1989). The specific activity of the enzyme following chromatography on Bio-Gel P-100 was 14,700 nmol 4 MU/min/mg TSP, comparable to that of the enzyme isolated from several mammalian sources (Kakkis et al., 1994; Ohshita et al., 1989, Schuchman et al., 1984). The overall recovery from transformed WT and cgl seeds is summarized in Table 1. The results illustrate that plant-produced human IDUA displays specific activity comparable to that of mammalian systems.

### EXAMPLE 4

### Effects of Other Treatments on Recombinant Protein Production in Stably Transformed Tobacco Leaves

### Some Enhancement of Human α-Iduronidase is Achieved by Treatments Designed to Increase Endogenous ABA Levels

Some treatments (FIG. 10) show an enhancement of human IDUA activities. Accordingly, in some embodiments, stress treatments (in place of or in addition to exogenous ABA) can induce expression of heterologous genes in. In particular, NaCL treatments may for example be applied to tissue-cultured transgenic plant cells expressing recombinant therapeutic proteins.

### REFERENCES CITED

The following documents, which do not necessarily constitute prior art, are incorporated herein by reference.
Bardor, M., Faveeuw, C., Fitchette, A. C., Gilbert, D., Galas, L., Trottein, F., Faye, L. and Lerouge, P. Immunoreactivity in mammals of two typical plant glyco-epitopes, core alpha (1,3)-fucose and core xylose. Glycobiology 13, 427-434 (2003).
Bonetta, D. and McCourt, P. (1998) Genetic analysis of signal transduction. Trends Plant Sci. 6: 231-235.
Brady, R. O. (2003) Enzyme replacement therapy: conception, chaos and culmination. Phil. Trans. R. Soc. Lond. B, 358, 915-919.
Carson, C. B., Hattori, T., Rosenkrans, L., Vasil, V., Vasil, I. K., Peterson, P. A. and McCarty, D. R. (1997) The quiescent/colorless alleles of viviparous1 show that the conserved B3 domain of VP1 is not essential for ABA-regulated gene expression in the seed. Plant J. 12:1231-1240.
Choi, S.-B., Wang, C., Muench, D. G., Ozawa, K., Franceschi, V. R., Wu, Y. and Okita, T. W. (2000) Messenger RNA targeting of rice seed storage proteins to specific ER subdomains. Nature 407: 765-767.
Clements, P. R., Brooks, D. A., Saccone, G. T. P. and Hopwood, J. J. (1985) Human α-L-iduronidase. 1. Purification, monoclonal antibody production, native and subunit molecular mass. Eur. J. Biochem. 152: 21-28.
Clements, P. R., Brooks, D. A., McCourt, P. A. G. and Hopwood, J. J. (1989) Immunopurification and characterization of human α-L-iduronidase with the use of monoclonal antibodies. Biochem. J. 259: 199-208.
Crofts, A. J., Washida, H., Okita, T. W., Ogawa, M., Kumamaru, T. and Satoh, H. (2004) Targeting of proteins to endoplasmic reticulum-derived compartments in plants. The importance of RNA localization. Plant Physiol. 136: 3414-3419.
Daniell, H., Streatfield, S. J. and Wycoff, K. (2001) Medical molecular farming: production of antibodies, biopharmaceuticals and edible vaccines in plants. Trends Plant Sci. 6: 219-226.
Datla, R. S. S., Bekkaoni, F., Hammerlindl, J. K., Pilate, G., Dunstan, D. I. and Crosby, W. L. (1993) Improved high-level constitutive expression in plant using an AMV RNA4 untranslated leader sequence. Plant Sci. 94: 139-149.
Depicker, A, Stachel S., Dhaese, P, Zambrinski P. and Goodman, H. M. (1982) Nopaline synthase: transcript mapping and DNA sequence. J. Mol. Appl. Genet. 1: 561-573.
Desnick R. J. and Schuchman, E. H. (2002) Enzyme replacement and enhancement therapies: Lessons from lyosomal disorders. Nature Rev. Genet. 3: 954-966.
Ezcurra, I., Wycliffe, P., Nehlin, L., Ellerstrom, M. and Rask, L. (2000) Transactivation of the Brassica napus napin promoter by ABI3 requires interaction of the conserved B2 and B3 domains of ABI3 with different cis-elements: B2 mediates activation through an ABRE, whereas B3 interacts with an RY/G-box. Plant J. 24: 57-66.
Finkelstein, R. R., Gampala, S. S, and Rock, C. D. (2002) Abscisic acid signaling in seeds and seedlings. Plant Cell 14 Suppl: S15-S45.
Fischer, R., Stoger, E., Schillberg, S., Christou, P. and Twyman, R. M. (2004) Plant-based production of biopharmaceuticals. Curr. Opin. Plant Biol. 7: 152-158.
Giddings, G., Allison, H., Brooks, D. and Carter, A. (2000) Transgenic plants as factories for biopharmaceuticals. Nature Biotechnol. 18: 1151-1155.
Giraudat, J., Hauge, B. M., Valon, C., Smalle, J., Parcy, F. and Goodman, H. M. (1992) Isolation of the Arabidopsis ABI3 gene by positional cloning. Plant Cell 4: 1251-1261.
Giraudat, J., Parcy, F., Bertauche, N., Gosti, F., Leung, J., Morris, P. C., Bouvier-Durand, M. and Vartanian, N. (1994) Current advances in abscisic acid action and signalling. Plant Mol. Biol. 26: 1557-1577.
Gomez, L. and Chrispeels, M. J. (1994) Complementation of an Arabidopsis thaliana mutant that lacks complex asparagine-linked glycans with the human cDNA encoding N-acetylglucosaminyltransferase I. Proc. Natl. Acad. Sci. USA 91: 1829-1833.
Gomord, V. and Faye, L. (2004) Posttranslational modification of therapeutic proteins in plants. Curr. Opin. Plant Biol. 7: 171-181.
Goossens, A., Ardiles Diaz, W., De Keyser, A., Van Montagu, M. and Angenon, G. (1995) Nucleotide sequence of an arcelin 5-I genomic clone from wild Phaseolus vulgaris (accession no. Z50202). Plant Physiol. 109: 722.
Goossens, A., Dillen, W., De Clercq, J., Van Montagu, M. and Angenon, G. (1999) The arcelin-5 gene of Phaseolus vulgaris directs high seed-specific expression in transgenic Phaseolus acutifolius and Arabidopsis plants. Plant Physiol. 20: 1095-1104.
Hill, A., Nantel, A., Rock, C. D. and Quatrano, R. S. (1996) A conserved domain of the viviparous-1 gene product enhances the DNA binding activity of the bZIP protein EmBP-1 and other transcription factors. J. Biol. Chem. 271: 3366-3374.
Hood, E. E. (2004) Where, oh where has my protein gone? Trends Biotechnol. 22: 53-55.
Horn, M. E., Woodard, S. L. and Howard, J. A. (2004) Plant molecular farming: systems and products. Plant Cell Rep. February 28 [Epub ahead of print].
Jiang, L. and Sun, S. S. M. (2002) Membrane anchors for vacuolar targeting: Application in plant bioreactors. Trends Biotechnol. 20: 99-102.
Jones, H. D., Kurup, S., Peters, N. C. and Holdsworth, M. J. (2000) Identification and analysis of proteins that interact with Avena fatua homologue of the maize transcription factor VIVIPAROUS1. Plant J. 21: 133-142.
Kermode, A. R. and Finch-Savage, W. (2002) Desiccation sensitivity in orthodox and recalcitrant seeds in relation to development. In: Desiccation and Plant Survival (Black, M. and Pritchard H., Eds.), pp. 149-184. CAB I, Oxon, UK.
Koornneef, M., Bentsink, L. and Hilhorst, H. (2002) Seed dormancy and germination. Curr. Opin. Plant Biol. 5: 33-36.
Kroj, T., Savino, G., Valon, C., Giraudat, J. and Parcy, F. (2003) Regulation of storage protein gene expression in Arabidopsis. Devel. 130: 6065-6073.
Kurup, S., Jones, H. D. and Holdsworth, M. J. (2000) Interactions of the developmental regulator ABI3 with proteins identified from developing Arabidopsis seeds. Plant J. 21:143-155.
Lazarova, G., Zeng, Y. and Kermode, A. R. (2002) Cloning and expression of an ABSCISIC ACID-INSENSITIVE 3 (ABI3) gene homologue of yellow-cedar (Chamaecyparis nootkatensis). J. Exp. Bot. 53: 1219-1221.
Li, G., Chandrasekharan, M. B., Wolffe, A. P. and Hall, T. C. (2001) Chromatin structure and phaseolin gene regulation. Plant Mol. Biol. 46: 121-129.
Ma, J. K., Drake, P. M. and Christou, P. (2003) The production of recombinant pharmaceutical proteins in plants. Nat. Rev. Genet. 4: 794-805.
McCarty, D. R., Hattori, T., Carson, C. B., Vasil, V., Lazar, M. and Vasil, I. K. (1991) The Viviparous-1 developmental gene of maize encodes a novel transcriptional activator. Cell 66: 895-905.
McCarty, D. R. (1995) Genetic control and integration of maturation and germination pathways in seed development. Ann. Rev. Plant Physiol. Plant Mol. Biol. 46: 71-93.
Nakamura, S., Lynch, T. J. and Finkelstein, R. R. (2001) Physical interactions between ABA response loci of Arabidopsis. Plant J. 26: 627-635.
Nambara, E., Hayama, R., Tsuchiya, Y., Nishimura, M., Kawaide, H., Kamiya, Y. and Naito, S. (2000) The role of ABI3 and FUS3 loci in Arabidopsis thaliana on phase transition from late embryo development to germination. Dev. Biol. 220: 412-423.
Neufeld, E. F., and Muenzer, J. (2001) The mucopolysaccharidoses. In: Metabolic and Molecular Bases of Inherited Disease, 8th edn. Vol. III. (Scriver, C. R., Beaudet, A. L., Sly, W. S., Valle, D., Childs, B., Kinzler, K. W. and Vogelstein B., Eds.), pp. 3421-3452, McGraw-Hill, Medical Publishing Division, New York.
Obermeyer, G., Gehwolf, R., Sebesta, W., Hamilton, N., Gadermaier, G., Ferreira, F., Commandeur, U., Fischer, R. and Bentrup, F.-W. (2004) Over-expression and production of plant allergens by molecular farming strategies. Methods 32: 235-240.
Paek, N. C., Lee, B. M., Gyu, B. D., and Smith, J. D. (1998) Inhibition of germination gene expression by Viviparous-1 and ABA during maize kernel development. Mol. Cells 8: 336-342.
Parcy, F. and Giraudat, J. (1997) Interactions between the ABI1 and the ectopically expressed ABI3 genes in controlling abscisic acid responses in Arabidopsis vegetative tissues. Plant J. 11: 693-702.
Parcy, F., Valon, C., Raynal, M., Gaubier-Comella, P., Delseny, M. and Giraudat, J. (1994) Regulation of gene expression programs during Arabidopsis seed development: roles of the ABI3 locus and of endogenous abscisic acid. Plant Cell 6: 1567-1582.
Parcy, F., Valon, C., Kohara, A., Misera, S, and Giraudat, J. (1997) The ABSCISIC ACID-INSENSITIVE3, FUSCA3, and LEAFY COTYLEDON1 loci act in concert to control multiple aspects of Arabidopsis seed development. Plant Cell 9: 1265-1277.
Rohde, A., Kurup, S., and Holdswoth, M. (2000) ABI3 emerges from the seed. Trends Plant Sci. 5: 418-419.
Schillberg, S., Fischer, R. and Emans, N. (2003) Molecular farming of recombinant antibodies in plants. Cell Mol. Life. Sci. 60: 433-445.
Scott, H. S., Anson D. S., Orsborn, A. M. and Nelson, P. V. (1991) Human alpha-L-iduronidase: cDNA isolation and expression. Proc. Natl. Acad. Sci. USA 88: 9695-9699.
Scott, H. S., Bunge, S., Gal, A., Clarke, L. A., Morris C. P. and Hopwood J. J. (1995) Molecular genetics of Mucopolysaccharidosis type I: Diagnostic, clinical, and biological implications. Human Mutat. 6: 288-302.

Scott, H. S., Guo, S. H., Hopwood, J. J. and Morris, C. P. (1992) Structure and sequence of the human alpha-L-iduronidase gene. Genomics 13: 1311-1313.

Sly, W. S. (2000) The missing link in lysosomal enzyme targeting. J. Clin. Invest. 105: 563-564.
Soderman, E. M., Brocard, I. M., Lynch, T. J. and Finkelstein, R. R. (2000) Regulation and function of the Arabidopsis ABA-insensitive4 gene in seed and abscisic acid response signaling networks. Plant Physiol. 124: 1752-1765.
Stoger, E., Ma, J. K.-C., Fischer, R. and Christou, P. (2005). Sowing the seeds of success: pharmaceutical proteins from plants. Curr. Opin. Biotechnol. 16: 1-7.
Suzuki, M., Kao, C. Y., Cocciolone, S. and McCarty, D. R. (2001) Maize VP1 complements Arabidopsis abi3 and confers a novel ABA/auxin interaction in roots. Plant J. 28: 409-418.
Twyman, R. M., Stoger, E., Schillberg, S., Christou, P. and Fischer, R. (2003) Molecular farming in plants: Host systems and expression technology. Trends Biotechnol. 21: 570-578.
Von Schaewen, A., Sturm, A., O'Neill, J. and Chrispeels, M. J. (1993) Isolation of a mutant Arabidopsis that lacks N-Acetylglucosaminyl Transferase I and is unable to synthesize Golgi-mediated complex N-linked glycans. Plant Physiol. 102: 1109-1118.
Voss, A., Niersbach, M., Hain R., Hirsch, R., Liao Y. C., Kreuzaler, F. and Fischer, R. (1995) Reduced virus infectivity in N. tabacum secreting a TMV-specific full-size antibody. Mol. Breed. 1: 39-50.
Werber, Y. (2004) Lysosomal storage diseases market. Nature Rev. 3: 9-10.
Zeng, Y. and Kermode, A. R. (2005) A gymnosperm ABI3 gene functions in a severe abscisic acid-insensitive mutant of Arabidopsis (abi3-6) to restore the wild-type phenotype and demonstrates a strong synergistic effect with sugar in the inhibition of post-germinative growth. Plant Mol. Biol., in press.
Zeng, Y., Raimondi, N. and Kermode, A. R. (2003) Role of an ABI3 homologue in dormancy maintenance of yellow-cedar seeds and in the activation of storage protein and Em gene promoters. Plant Mol. Biol. 51: 39-49.
Zhao, K. W., Faull, K. F., Kakkis, E. D. and Neufeld, E. F. (1997) Carbohydrate structures of recombinant human α-L-iduronidase secreted by Chinese hamster ovary cells. J. Biol. Chem. 272: 22758-22765.

### CONCLUSION

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

TABLE 1
Purification summary for IDUA derived from
transgenic WT (a) and cg/mutant (b) seeds

| | | | | |
|---|---|---|---|---|
| Step | Protein (mg) | Units/mg³ | Total units | Yield |

a

| | | | | |
|---|---|---|---|---|
| Crude^{b} | 170 | 6.3 | 1,072 | 100 |
| ConA^{c} | 20 | 29 | 586 | 55 |
| Ab^{d} | 0.03 | 9,033 | 271 | 25 |
| BGel^{e} | 0.01 | 14,700 | 147 | 14 |

b

| | | | | |
|---|---|---|---|---|
| Crude^{b} | 270 | 2.04 | 544 | 100 |
| ConA^{c} | 27 | 8 | 216 | 40 |
| Ab^{d} | 0.02 | 2,700 | 54 | 10 |
| BGel^{e} | 0.006 | 7,800 | 47 | 8.6 |

^{a}Units are nmoles 4MU formed per minute.
^{b}Crude: clarified lysate (lipid removed).
^{c}ConA: combined elution fractions from concanavalin A/Sepharose column.
^{d}Ab: combined elution fractions from antibody column.
^{e}BGel: combined elution fractions from Bio-Gel P100 column.

1. An expression system for heterologous protein expression in vegetative plant tissues, the expression system comprising a host plant cell having a recombinant genome, wherein the plant cell is maintained under protein expressing conditions and expresses an ABI3 transcription factor, wherein the recombinant genome comprises, in operative linkage:
a) an integrated expression promoter responsive to the ABI3 transcription factor, having at least 70% identity when optimally aligned to a plant seed gene promoter selected from the group consisting of an arcelin gene promoter, a vicilin gene promoter and a napin gene promoter;
b) an integrated 5′ untranslated region having at least 70% identity when optimally aligned to a 5′ untranslated plant seed gene region of an ABA responsive plant seed gene or an ABI3 responsive plant seed gene;
c) an integrated plant secretion signal peptide coding sequence;
d) an integrated heterologous protein coding region, encoding a recombinant protein, in an open reading frame with the signal peptide coding sequence; and,
e) a 3′ untranslated region having at least 70% identity when optimally aligned to a 3′ untranslated plant gene region having a polyadenylation signal.

2. The expression system of claim 1, wherein the protein expressing conditions comprise providing an exogenous abscisic acid to the cell.

3. The expression system of claim 1, wherein the protein expressing conditions comprise conditions that elevate the concentration of endogenous abscisic acid in the cell.

4. The expression system of claim 3, wherein the conditions that elevate the concentration of endogenous abscisic acid in the cell comprise a sodium chloride concentration that elevates the concentration of endogenous abscisic acid in the cell.

5. The expression system of any one of claim 1, wherein the heterologous protein is an alpha-L-iduronidase protein.

6. The expression system of any one of claim 1, wherein the activity of a N-acetylglucosaminyl transferase in the cell is below a threshold level, the expression of the heterologous protein being greater below the threshold level than above the threshold level.

7. The expression system of claim 6, wherein an N-acetylglucosaminyl transferase I activity in the cell is reduced, compared to the N-acetylglucosaminyl transferase I activity in a non-recombinant host cell.

8. The expression system of claim 1, wherein the recombinant genome further comprises an ER retention signal sequence, in operative linkage with the expression promoter and in the open reading frame.

9. The expression system of claim 8, wherein the ER retention signal sequence encodes a carboxy terminal SEKDEL sequence on the heterologous protein.

10. The expression system of claim 1, wherein the plant tissues further comprise plant seed tissues.

11. The expression system of claim 1, wherein the heterologous protein is a human protein.

12. The expression system of claim 1, wherein the 3′ untranslated region is of an ABA responsive plant seed gene or an ABI3 responsive plant seed gene.

13. The expression system of claim 1, wherein the recombinant genome further comprises a signal peptide coding sequence, in operative linkage with the expression promoter and in the open reading frame.

14. The expression system of claim 1, wherein the expression promoter has at least 95% identity when optimally aligned to the arcelin gene promoter, the vicilin gene promoter or the napin gene promoter.

15. The expression system of claim 1, wherein the 5′ untranslated plant seed gene region comprises a 5′ untranslated region from an arcelin gene, a vicilin gene or a napin gene.

16. The expression system of claim 1, wherein the 3′ untranslated region comprises a 3′ untranslated region from an arcelin gene, a vicilin gene or a napin gene.

17. The expression system of claim 1, wherein the expression promoter is a Phaseolus vulgaris arcelin gene promoter.

18. The expression system of claim 1, wherein the 5′ untranslated region is a Phaseolus vulgaris arcelin gene 5′ untranslated region.

19. The expression system of claim 1, wherein the 3′ untranslated region is a Phaseolus vulgaris arcelin gene 3′ untranslated region.

20. The expression system of claim 1, wherein the ABI3 transcription factor is expressed by a recombinant transcription factor gene having a constitutive promoter operatively linked to an ABI3 transcription factor coding sequence.

21. A recombinant plant expression vector comprising in operative linkage:
a) an expression promoter having at least 70% identity when optimally aligned to a plant seed gene promoter selected from the group consisting of an arcelin gene promoter, a vicilin gene promoter and a napin gene promoter;
b) a 5′ untranslated region having at least 70% identity when optimally aligned to a 5′ untranslated plant seed gene region of an ABA responsive plant seed gene or an ABI3 responsive plant seed gene;
c) a plant signal peptide coding sequence;
d) a heterologous protein coding region, encoding a recombinant protein, in an open reading frame with the signal peptide coding sequence; and,
e) a 3′ untranslated region having at least 70% identity when optimally aligned to a 3′ untranslated plant gene region having a polyadenylation signal.

22. A method of expressing a heterologous protein in vegetative plant tissues, the tissues comprising recombinant host plant cells having recombinant genomes, the method comprising maintaining the plant cells under protein expressing conditions, wherein the plant cells express an ABI3 transcription factor and the recombinant genomes comprise, in operative linkage:
a) an integrated expression promoter responsive to the ABI3 transcription factor, having at least 70% identity when optimally aligned to a plant seed gene promoter selected from the group consisting of an arcelin gene promoter, a vicilin gene promoter and a napin gene promoter;
b) an integrated 5′ untranslated region having at least 70% identity when optimally aligned to a 5′ untranslated plant seed gene region of an ABA responsive plant seed gene or an ABI3 responsive plant seed gene;
c) an integrated plant secretion signal peptide coding sequence;
d) an integrated heterologous protein coding region, encoding a recombinant protein, in an open reading frame with the signal peptide coding sequence; and,
e) a 3′ untranslated region having at least 70% identity when optimally aligned to a 3′ untranslated plant gene region having a polyadenylation signal.


SEQUENCE LISTING (39 sequences)

SEQ ID NO: 1 - protein, 6 aa, Artificial Sequence
  retention sequence
SEKDEL

SEQ ID NO: 2 - DNA, 11 bp, Artificial Sequence
  promoter
gtacgtggcgc

SEQ ID NO: 3 - protein, 115 aa, Artificial Sequence
  B3 DNA Binding Domain
LFQKELTPSDVGKLNRLVIPKKYAVKYMPFISDDQSEKETSEGVEDVEVVFYDRAMRQWKFRYCYWRSSQSFVFTRGWNGFVKEKNLKEKDIIVFYTCDVPNNVKTLEGQSKTFL

SEQ ID NO: 4 - protein, 105 aa, Artificial Sequence
  B3 DNA Binding Domain
LFEKTLSASDAGRIGRLVLPKACAEAYFPPISQSEGIPLKIQDVRGREWTFQFRYWPNNNSRMYVLEGVTPCIQSMMLQAGDTVTFSRVDPGGKLIMGSRKAANA

SEQ ID NO: 5 - protein, 105 aa, Artificial Sequence
  B3 DNA Binding Domain
LLQKVLKQSDVGNLGRIVLPKKEAETHLPELEARDGISLAMEDIGTSRVWNMRYRFWPNNKSRMYLLENTGDFVKTNGLQEGDFIVIYSDVKCGKYLIRGVKVRQ

SEQ ID NO: 6 - protein, 108 aa, Artificial Sequence
  B3 DNA Binding Domain
MFEKVVTPSDVGKLNRLVVPKHYAEKYFPLGPAARTSPAGTVLCFEDARGGDSTWRFRYSYWSSSQSYVITKGWSRYVRDKRLAAGDTVSFCRAGARLFIDCRKRAAS

SEQ ID NO: 7 - protein, 106 aa, Artificial Sequence
  B3 DNA Binding Domain
FFCKTLTASDTSTHGGFSVPRRAAEKLFPPLDYSAQPPTQELVVRDLHENTWTFRHIYRGQPKRHLLTTGWSLFVGSKRLRAGDSVLFIRDEKSQLMVGVRRANRQ

SEQ ID NO: 8 - protein, 94 aa, Artificial Sequence
  B3 DNA Binding Domain
EFKITIRKSYLKFLAIPKHFVDDHIPNKSKIFTIRHPNGGSWKVLCLVREIRTIFSGGYSKLAREFPLMVGDKCTFKLIKPFEFVLLTSKKNRE

SEQ ID NO: 9 - protein, 102 aa, Artificial Sequence
  B3 DNA Binding Domain
FFTALIAKSHLHPKFQMWIPPRFQHRLAEPEARTAAVLHSGGKSWATSYCGHLKMKKLDAGWSEFAVDNRLLVGDACVFELVAMGAAGGLEFQVQILRGGLP

SEQ ID NO: 10 - protein, 95 aa, Artificial Sequence
  B3 DNA Binding Domain
SFTKPMLQSHVTGGFWLGLPLPFCKAHMPKRDVIMTLVDEEEEESQAKYLAQKNGLSGGWRGFAIDHQLVDGDAVVFHLIARTTFKVYIIRVNDD

SEQ ID NO: 11 - protein, 100 aa, Artificial Sequence
  B3 DNA Binding Domain
HFVRNITRGSLQKLELPLTFLRSNGIELEEDIELCDESGKKWPLKILNHDRGFKFSHESWLCFCKSHEMILTNKCLFEFIVPSNGRCSEILVRIVSGRLP

SEQ ID NO: 12 - protein, 96 aa, Artificial Sequence
  B3 DNA Binding Domain
DFLKIFNSHEDSQLLVIPRSYNRYYPNPLPQTAVLKNPEGRFWNVQWTKSQEVIISLQEGWVKFVKDNGLIDRDFLLFTYDGSRSFWVRIHRNGLP

SEQ ID NO: 13 - protein, 101 aa, Artificial Sequence
  B3 DNA Binding Domain
YCLLGLTASNLRLNRVSFTKHFSRANGLTKRCCMIDLMNLSGESWTLGLRHNKRTGQAFIRGRWRSFCHANELKPGSFYRFKLVRNGTRPLLQLCFKVIPQ

SEQ ID NO: 14 - protein, 94 aa, Artificial Sequence
  B3 DNA Binding Domain
TGLYIILVNVGVVQMIPAEFFSTYVEGKNHQSTKLKLTSDAFDRTWEVKLNGRRFAGGWENFSTVHSLQDDDVVIFREIGDMTFHVTASGRSFC

SEQ ID NO: 15 - protein, 93 aa, Artificial Sequence
  B3 DNA Binding Domain
AFFIIDLSGQKSNPIIPTEFIWNHFNGKIQSTNMKLTSDASDRNWDVKLDGARFAGGWKDFSVSHSVRDDDLLSFRHDGGMVFHVSPFGRSFS

SEQ ID NO: 16 - protein, 100 aa, Artificial Sequence
  B3 DNA Binding Domain
ILTFDLKPYVFRSCQFFLPASFARENGIVEAGEVTVLNKDGIEWKSHLVNIKGRDQFYNRGCQDFFVANGVKNVGDPFTLEVIRGGPSPILKICSKVKQA

SEQ ID NO: 17 - protein, 93 aa, Artificial Sequence
  B3 DNA Binding Domain
HFFQPLLPGFKSHINIPVKFFSKYIKGKHEGKTVKLRSDSSKRTWKVKIEGHTLTDGWKEFVEAHDLRISDFVIFKHKGDMFFDVTALGSSCW

SEQ ID NO: 18 - protein, 99 aa, Artificial Sequence
  B3 DNA Binding Domain
CITRGWRHFCDENGKKYLSRRFLKNNGLGEPKMVTLVGTDGTRILANLLRESTGRMSLGRGWVDFAKANRLKIGEYFTLESIWENDSPILSLYGTNTSK

SEQ ID NO: 19 - protein, 105 aa, Artificial Sequence
  B3 DNA Binding Domain
FLIVKYTPSRETTGQLSLPVSFTRNNSINKTGEVILLNQDGRKWSSYLQITGLGRGAGSEWFYLRRGWREMCEANGVGVNDSFKLELVWEGANPMFKFCSKIENH

SEQ ID NO: 20 - protein, 86 aa, Artificial Sequence
  B3 DNA Binding Domain
LFQLTFLTGDKPILTLDDEFISSHTKVLLISDASDKIWEVKLDGNRLAGGWEEFAAVNNFSEGNVLVFRHNGEEIFHVAVSSESDD

SEQ ID NO: 21 - protein, 99 aa, Artificial Sequence
  B3 DNA Binding Domain
FVTFTPEDIRDCILILPSQFIKANGINNLGEITLLGQNRMKWFAYLLSMSKDGSLALGSGWKGICEANGVNTGEAFTLEYIDEQETAHKTSQCVGEDDA

SEQ ID NO: 22 - protein, 102 aa, Artificial Sequence
  B3 DNA Binding Domain
ILFGESVFSRLLYLCLYLPQDLTSSVGLERKYREIVVTDERERRSWALDLRFNKSSDTFYISRGWRSFCDENGKKPGGVFVFKLVGNRETPVLSFCSTESIN

SEQ ID NO: 23 - protein, 720 aa, Arabidopsis thaliana
MKSLHVAANAGDLAEDCGILGGDADDTVLMDGIDEVGREIWLDDHGGDNNHVHGHQDDDLIVHHDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMMENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKCGKYLIRGVKVRQPSGQKPEAPPSSAATKRQNKSQRNINNNSPSANVVVASPTSQTVK

SEQ ID NO: 24 - protein, 651 aa, A. thaliana
DPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAATKRQNKSQRNINNNSPSANVVVASPTSQTVK

SEQ ID NO: 25 - protein, 681 aa, P. balsamifera
DVSIFYEDFPPLPDFPCMSSSSSSSSTPAPVNAITSSSSSSCSSSASSSSSAAAWAVLKSEAEEDVEKNHQHRNHCYHHNNNDDFNSQAMDDPVDVSTAALSSTCSMEVPQPPDQAMELGIECMDVMEDFGYIDLLESNDFFDPSSIFHPDEGLFEEFQMEQNEPQDQLQLQYYDEQAGNEEITKGKNDQEADHQGGRSDDLAMVFLDWLKSNKETVSADDLRRVKLKKTTIECAARRLGGGKEGMKQLLKLILQWVQTNHLQRRRMRESSSNVNLLYPYNQDPLQNQNPNPNSNLNCNPIPADHSNPCFTQSPWNVAPPPYLAADPATVMPGFSPMVGFMGDPFSNGSSNINGHPYGTPQDCNHMLQSYQTWPPSQFHSASHFNSFADNNLQSAQPQNPAFTGYGNQYPYQYVPANGDNRLTRLGSSATKEARKKRMARQRRFLSYHRNQNHHNIQHQNQGAGDPHERLSDDPNGAPTGQSNPGSWVYWPTAAGGGSASTTVDAPVDRPAMQAQTNNHRQAAAERRQGWKPEKNLRFLLQKVLKQSDVGSLGRIVLPKKEAETHLPELEARDGISIAMEDIGTSRVWNMRYRFWPNNKSRMYLLENTGDFVRTNGLQEGDFIVIYSDVKCGKYLIRGVKVRQPAGPKPENKRAGKSQRNSHANCPAAANNGSGSQKQTVK

SEQ ID NO: 26 - protein, 690 aa, A. thaliana
HVAANAGDLAEDCGILGGDADDTVLMDGIDEVGREIWLDDHGGDNNHVHGHQDDDLIVHHDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAATKRQNKSQRN

SEQ ID NO: 27 - protein, 768 aa, Prunus avium
HQDLHAGDLHHHHMKDVNIPISDGFGGGGAMEELEDQEDNLGVDPREMWLDDNDQETAFLADVNDPSIFYNDHFPPLPDFPCMSSSSSSSSTPAPVKPVTSSSTSSSISSSSSAASWAILRSDAEEDGERRQQQHHNSYNHRYQYSQVDDQAVDAHALSSTASMEISQPSDLGREGGAIDCMGAMETFGYTDLFESNEFFDLSSIFQSDSLLMEQFQQDDDHQQLLTPHQLQDPNEATAIIPQQQQQQEVAVRDEENNKKDDQNENKEPDDMAMVFLEWLRSNRETVSAEDLRSVKIKKSTIECAARRLGGGKEAMKQLLKLVLEWVQTNHLQKRRSNSLTTKDANIVAQQQQYHDPFQNPNPNTSPRVLEPNPSCSFTQTPWMAPPPHAAYDHAGESLSPLRPRRPPPAAYPSMMGYIAPDQYVNGPGPYQPSPEYHHMIDSGQPTWPSSPFGMGTAHYGSFPDNNIHLAPPPQHRPQAFAGYGSQYQPYQYFPGNGEHQLMRLGSSATKEARKKRMARQRRLVSHHRHGHHHQQQHLNAQMPDHLLHQQHTRLVGNAANLNCANSVPLQANPGNWFYWATATAAPSPSPAMMPSITPEAAPPPPVQQMDRPASTQAQNYNQGRSAAQERQERRQGWKSEKNLKFLLQKVLKQSDVGSLGRIVLPKKEAETHLPELEARDGISIPMEDIGTSRVWNMRYRYWPNNKSRMYLLENTGDFVRANGLQEGDFIVIYSDVKCNKYMIRGVKVRQAGPKSEGNKRPGKSQRN

SEQ ID NO: 28 - protein, 631 aa, A. thaliana
HDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAATKRQNKSQRN

SEQ ID NO: 29 - protein, 665 aa, Psophocarpus tetragonolobus
NDASMFYADFPPLPDFPCMSSSSSSSSAPPLPAKTMACSTTTTTTSSSSSSSSWVMLRSDVEEDAEKNHCNHYMHDQLDATALSSTASMEISQQHNPDPALGGTVGECMEDVMDTFGYMELLESNDFFDPASIFQEDNEDPLVEFGTLEEQVPLHDEQHAMVHQKGKADEEDHQVPVCEEIHGEEEGGDGVGVVDDEMSNVFLEWLKSNKDSVSANDLRNVKLKKATIESAARRLGGGKEAMKQLLKLILEWVQTSHLQNKRRKENSGSISTVLQGQFQDPSVQNTHTGSFAPEPNSCFNNQTPWLSPQPFGTDQNPLMVPSQQFPQPMVGYVGDPYTSGAASNNITATHNHNNNPYQPGAEQYHMLESAHSWPHSQFNVASHYGQSFGENGLFPHGGFGGYGNNQYPYQFFHGPGDRLMRLGPSATKEARKKRMARQRRFLSHHRNHNGNHQQNQGNDPHATLGGDNCTNVVAAPHANHAANWMYWQAMTAGVAGTLGPVVPAEPPAGQPVVDRSTIHTQNCHQSRVASDRRQGWKPEKNLRFLLQKVLKQSDVGSLGRIVLPKKEAETHLPELEARDGISITMEDIGTSRVWNMRYRYWPNNKSRMYLLENTGDFVRANGLQEGDFIVIYSDVKCGKYMIRGVKVRQQGVKPETKKGGKSQKN

SEQ ID NO: 30 - protein, 631 aa, A. thaliana; identical to SEQ ID NO: 28

SEQ ID NO: 31 - protein, 676 aa, Phaseolus vulgaris
NEASMFYADFPPLPDFPCMSSSSSSSSAAPLPLKTTTCSTTTTATTATSSSSSSSSWAVLKSDVEEDVEKNHCNGSMQDQFDATALSSTASMGISQQQNPDPGLGGSVGECMEDVMDTFGYMELLEANDFFDPASIFQNEESEDPLIEFGVLEEQVSLQEEQHEMVHQQENTEEDRKVPVCGVIKGEEEGGGGGGGRVVDDEMSNVFLEWLKSNKDSVSANDLRNVKLKKATIESAAKRLGGGKEAMKQLLKLILEWVQTSHLQNKRRKENGSNNSNALQAQFQDPSAQTKENAHTSGSFAPESNSCFNNQTPWLNPQTFGTDQAPVMVPSQPYSQPVVGYVGDPYTSGSAPNNITVNHNHNNNPYQPGTDQYHMLESAHSWPHSQFNVASHYSQSYGENGLFTHGGFGGYANNQYPYQFFHGPGDRLMRLGPSATKEARKKRMARQRRFLSHHRNHNGNHLQNQGSDPHARLGNDNCTTGLVAPHQPNSAAANWMYWQAMTGGPGGPLAPVVPADPLAGQTVVDRTTMHTQNSHQNRAASDRRQGWKPEKNLRFLVQKVLKQSDVGKLGEIVLPKKEAETHLPELEARDGISITMEDIGTSRVWNMRYRYWPNNKSRMYLLENTGDFVRANGLQEGDFIVIYSDVKCGKYMIRGVKVRQQGVKPETKKAGKSQKN

SEQ ID NO: 32 - protein, 623 aa, A. thaliana
HDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAATK

SEQ ID NO: 33 - protein, 673 aa, Phaseolus vulgaris
NEASMFYANFPPLPDFPCTSSSSSSSSAAPLPLKTTTCSTTTTATTATSSSSSSSSWAVLKSDVEEEDVEKNHCNGSMQDQFDATALSSTASMEISQQQNPDPGLGGSVGECMEDVMDTFGYMELLEANDFFDPASIFQNEESEDPLIEFGVLEEQVSLQEEQHEMVHQQENTEEDRKVPVCEVIKGEEEGGGGGGGRVVDDEMSNVFLEWSKSNKDSVSANDLRNVKLKKATIESAAKRLGGGKEAMKQLLKLILEWVQTSHLQNKRRKENGSNALQATFQDPSAQTKENAHTSGSFAPESNSCFNNQTPWLNPQTFGTDQAPVMVPSQPYSQPVAGYVGDPYTSGSAPNNITVNHNHNNNPYQPGTDQYHMLESAHSWPHSQFNVASHYSQSYGENGLFTHGGFGGYAITRYPYQFFHGPGDRLMRLGPSATKEARKKRMARQRKFLSHHRNQNGNHLQNQGSDPHARLGNDNCTTGLVAPHQPNSAAANWMYWQAMTGGPAGPLAPVVPADPLAGQTVVDRTTMHTQNSHQNRAASDRRQGWKPEKNVRFLGQKVLKQSDVGKLGRIVLPKKEAETHLPELEARDGISITMEDIGTSRVWNMRYRYWPNNKSRMYMLENTGDFVRANGLQEGDFIVIYSDVKCGKYMIRGVKVRQQGVKPETKPAGKSQK

SEQ ID NO: 34 - protein, 575 aa, A. thaliana
WAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAATKRQNKSQ

SEQ ID NO: 35 - protein, 605 aa, Pisum sativum
WAVLKSEVEEDHHHGEKMKSCDNNHGFLNMHDPLDHHHHHHHGQHATTASIEIPQQQQELGVGDCMEDVMMDDTFGYMELLEANDFFDPASIFQTEGETPLVDDFTQEQEQVLVQHQQVPIVVHDDSETKLDLNFDGVGVNDGACDGVNDEMSNVFLEWLKSNKDSVSANDLRNVKLKKSTIESAARRLGGGKEGMKQLLKLILEWVQTSHLQNKRLKENNNNTTTSNVVPQQPLPQFKDLCPNQNTTNTCFNQTSWMDQTQTPLVVPPQQFSQAMVGVGYVGDIHYTNGSVSNSLYQQGSTNEYHQFNVVPNYNQPSFVDSNNNVVQPHGLSFGGYGNQYGSYQFFHGGGGDRLMRLGPSATKEARKKRMARQRRFVSHHRNHHQGSDSVARLGGGGGGGDNCTNGVGVGSHANQANWMYWQSMAGGKEASLAPVVRDEQTQPPVERDRTNNQTPNSHQGRNASDKKQGWKPEKNLKFLLQKVLKQSDVGSLGRIVLPKKEAETHLPELEARDGISITMEDIGTSRVWNMRYRYWPNNKSRMYLLENTGDFVKANGLQEGDFIVMYSDVKCGKFMIRGVKVRQQGAKPEAKKTGKAQKNQQQQQ

SEQ ID NO: 36 - protein, 628 aa, A. thaliana
DPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAATKRQNKSQ

SEQ ID NO: 37 - protein, 704 aa, Mesembryanthemum crystallinum
DPSSLFYAADDFPALPDFPCMSSSSSSSSAPAPKKPFASTATSSSASTATSSSWVADHEPSSSTAVSMDLVAPPPPQQSGGGGAGGEMGSMSVDDVDQCMDMMENFGCIDLLESGDICWDPSPLFGDGDGDESRQLLEEQQLERERERVEEEERAFEEFMLQGGESDSVVNVDDVVAGGNSNLDNTSNNNSKQQEHEQQHEQQGLVSSDDLAMVFFEWLKTNKEAISAEDLRNIKIKKSTIEAAAKRLGGGKEGMKQLLKLILQWVQNHHLHNKRESSTVSNNTCGAPVALVDQDHTNSTNNNNDNNNSIIADPNPNPNPNPTPPPLEQQASTTSSCFTTPPPATWLPAPQPQPFVGDPAAMVPAPPPMVGYMGSDPYSAGMAAYPPADYHQMMDTAPHSWAQTPSMQFGMGPQYGSFPDPSHAAQFGGYPAPYPGFYYHPGPGEGLMRLGSSATKEARKKRMARQRRFFTHHHRNHNHHQNQNQNNQMNNNLMVEQHGGVGNGNCGVAPHPSPAGNWVYWSHPPPLPPQVSHPVGGPPPMVGQMQGLERAAPSGNGFQRQGGVEKKQGWKSEKNLRFLLQKVLKQSDVGNLGRIVLPKKEAETHLPELEARDGIPIAMEDIGTSRVWNMRYRFWPNNKSRMYLLENTGDFVRSNGLQEGDFIVIYSDVKCGKYMIRGVKVRPQQQGAKAETTNKKSCKTQKTQ

SEQ ID NO: 38 - protein, 666 aa, A. thaliana
ILGGDADDTVLMDGIDEVGREIWLDDHGGDNNHVHGHQDDDLIVHHDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMLENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKLIRGVKVRQPSGQKPEAPPSSAA

SEQ ID NO: 39 - protein, 650 aa, Craterostigma plantagineum
IIGGDGDGENRELWLDGEDDDQDNLLLGVNEDSIFYTDFPSLPDFPCMSSSSSSSSNPKPIVSATTSSSAASSSWAAAMKSETAAAISSTASMEVQAPATLSDLDCCIDAMENFGYMDLIDVNEIWGGDDDPSDPVFAGDAEQTPAVIPAVDAPHQQLTFDSSPPPQENQRVITMAAQQSQENDGLTTLLQENSELAVIFFEWLKQNKDNISAEDMRSIKLRRSTIDNASKRLGSSKEGKIKLLKLILGWVEQCQLQKKKTNKVGGENSSQEISNSDPNQNPSHNFPYNPDCCFSTPPPPTPWLPPPPQETPAPASFPASYPQPPPMPMYPYDPYVNTVHHQIPPIPYPPPFVEYPPPPPMMEAQPWAAVAAPPYAVAAQPQFGAFPEPNFYACNPYQMCDLSGERFVKLGASATKEARKKRMARQRRLYSSHHRHGHHHHGHQIAPADANSMENHQNGGGDRSSPGNSSWMYNNVGASSNVVIQNVDSTQPSSGDKMAAQAQSSNQRLGSNDRRQQQQQQGLKTEKNLKFLLQKVLKQSDVGSLGRIVLPKKEAEIHLPELETRDGISVAMEDIGTSRVWNMRYRFWPNNKSRMYLLENTGDFVRLNGLQEGDFIVIYSDTKCGKYMIRGVKVRPGTKLESKKPAKKNA